SEQUENCE LISTING (31 sequences)

SEQ ID NO: 1 - DNA, 650 bp, Turicibacter sp.
  source 1..650: mol_type = other DNA
gcggcgaacgggtgagtaacacgtaggtgatctgcccatcagacggggacaacgattggaaacgatcgctaataccggataggacgaaagtttaaagatgctcctggcatcactgatggatgagcctgcggcgcattagctagttggtggggtaaaggcctaccaaggcgacgatgcgtagccgacctgagagggtgaacggccacactgggactgagacacggcccagactcctacgggaggcagcagtagggaatcttcggcaatgggcgaaagcctgaccgagcaacgccgcgtgaatgaagaaggccttcgggttgtaaaattctgttataagggaagaaaggtgataggaggaaatgactatcaattgacggtaccttatgagaaagccacggctaactacgtgccagcagccgcggtaatacgtaggtggcaagcgttatccggaattattgggcgtaaagagcgcgcaggtggttaattaagtctgatgtgaaagcccacggcttaaccgtggagggtcattggaaactggttgacttgagtgcagaagagggaagtggaattccatgtgtagcggtgaaatgcgtagagatatggaggaacaccagtggcgaaggcggcttcctggtctgcaactgacactg

SEQ ID NO: 2 - DNA, 650 bp, Brevibacillus sp.
  source 1..650: mol_type = other DNA
tagcggcggacgggtgagtaacacgtaggcaacctgcctctcagactgggataacatagggaaacttatgctaataccggataggtttttggatcgcatgatccgaaaagaaaagatggcttcggctatcactgggagatgggcctgcggcgcattagctagttggtggggtaacggcctaccaaggcgacgatgcgtagccgacctgagagggtgaccggccacactgggactgagacacggcccagactcctacgggaggcagcagtagggaattttccacaatggacgaaagtctgatggagcaacgccgcgtgaacgatgaaggtcttcggattgtaaagttctgttgttagggacgaataagtaccgttcgaatagggcggtaccttgacggtacctgacgagaaagccacggctaactacgtgccagcagccgcggtaatacgtaggtggcaagcgttgtccggatttattgggcgtaaagcgcgcgcaggcggctatgtaagtctggtgttaaagcccggngctcaaccccggttcgcatcggaaactgtgtagcttgagtgcagaagaggaaagcggtattccacgtgtagcggtgaaatgcgtagagatgtggaggaacaccagtggcgaaggcggctttc

SEQ ID NO: 3 - DNA, 376 bp, Anaerovorax sp.
  source 1..376: mol_type = other DNA
cagggatagccattggaaacgatgattaaaacctgataacaccatttggttacatgagcagatggtcaaagatttatcggcaaaggatgggcctgcgtctgattagctagttggtaaggtaacggcttaccaaggcgacgatcagtagccgacctgagagggtgaacggccacattggaactgagacacggtccaaactcctacgggaggcagcagtggggaatattgcacaatgggcgaaagcctgatgcagcaacgccgcgtgaaggaagaaggccttcgggtcgtaaacttctgtccttggggaagaagaactgacggtacccaaggaggaagccccggctaactacgtgccagcagccgcggtaatacgtag

SEQ ID NO: 4 - DNA, 378 bp, Clostridium sp.
  source 1..378: mol_type = other DNA; note = Clostridium XIVa
cagttagaaatgactgctaataccgcataagaccacaaagccgcatggctrwgtggtaaaaactccggtggtgtaagatgggcccgcgtctgattaggtagttggcggggtaacggcccaccaagccgacgatcagtagccgacctgagagggtgaccggccacattgggactgagacacggcccagactcctacgggaggcagcagtggggaatattgcacaatgggggaaaccctgatgcagcgacgccgcgtgagcgatgaagtatttcggtatgtaaagctctatcagcagggaagaaaatgacggtacctgactaagaagccccggctaactacgtgccagcagccgcggtaatacgtagggggcaagcgtta

SEQ ID NO: 5 - DNA, 610 bp, Roseburia sp.
  source 1..610: mol_type = other DNA
gggtgagtaacgcgtgaggaacctgccttggagtggggaataacagctggaaacagctgctaataccgcataatatatctgggccgcatggctctggatatcaaagatttatcgctctgagatggactcgcgtctgattagctggttggcggggtaacggcccaccaaggcgacgatcagtagccggactgagaggttggccggccacattgggactgagacacggcccagactcctacgggaggcagcagtggggaatattgggcaatgggcgcaagcctgacccagcaacgccgcgtgaaggaagaaggctttcgggttgtaaacttcttttgtcagggacgaagcaagtgacggtacctgacgaataagccacggctaactacgtgccagcagccgcggtaatacgtaggtggcannnnntatccggatttactgggtgtaaagggcgtgtaggcgggactgcaagtcagatgtgaaaaccacgggctcaacctgtggcctgcatttgaaactgtagttcttgagtactggagaggcagacggaattcctagtgtagcggtgaaatgcgtagatattaggaggaacaccagtggcgaaggcggtctg

SEQ ID NO: 6 - DNA, 611 bp, Oscillospira sp.
  source 1..611: mol_type = other DNA
gggtgagtaacgcgtgggcaacctgccccacacagggggataacagccggaaacggctgctaataccgcataagcgcacagcttcgcatggagcggtgtgaaaagcctttgggcggtgtgggatgggcccgcgtctgattagctggttggcggggcagcggcccaccaaggcgacgatcagtagccggcctgagagggtggacggccacattgggactgagacacggcccagactcctgcgggaggcagcagtggggaatattgcacaatgggggaaaccctgatgcagcgacgccgcgtgagcgaagaagtatttcggtatgtaaagctctatcagcagggaagaaaatgacggtacctgagtaagaagctccggctaaatacgtgccagcagccgcggtaatacgtatggagcaagcgttatccggatttactgggtgtaaagggagcgcaggcggccaggcaagtctgatgtgaaataccggggctcaaccccggaactgcattggaaactgtcaggctggagtgtcggagaggcaggcggaattcctggtgtagcggtgaaatgcgtagatatcaggaggaacaccagtggcgaaggcggcctgctg

SEQ ID NO: 7 - DNA, 376 bp, Anaerovorax sp.; identical to SEQ ID NO: 3
  source 1..376: mol_type = other DNA

SEQ ID NO: 8 - DNA, 200 bp, unidentified
  source 1..200: mol_type = other DNA; note = Eisenbergiella sp.
tacagggggataacacttagaaataggtgctaataccgcataagcgcacaggggcgcatgccccggtgtgaaaaactccggtggtatatgatggacccgcgtctgattagccagttggcagggtaacggcctaccaaagcgacratcartagccggcctgagagggcggacggccacattgggactgagacacggcccaa

SEQ ID NO: 9 - DNA, 378 bp, Clostridium sp.; identical to SEQ ID NO: 4
  source 1..378: mol_type = other DNA; note = Clostridium XIVa

SEQ ID NO: 10 - DNA, 214 bp, Anaerotruncus sp.
  source 1..214: mol_type = other DNA
taataccgcatgagactacagtactacatggtacagtggccaaaggagcaatccgctgaaagatgggctcgcgtccgattagatagttggcggggtaacggcccaccaagtcgacgatcggtagccggactgagaggttgaacggccacrttgggactgagacacggcccagactcctacgggaggcagcagtgagggatattggtcaatgggg

SEQ ID NO: 11 - DNA, 133 bp, Oscillibacter sp.
  source 1..133: mol_type = other DNA
agttggaaacgactgctaataccgcatgatacatttgggtcgcatggtctgaatgtcaaagatttatcgccgaaagatggcctcgcgtctgattagctagttggtggggtaacggcccaccaaggcgacgatc

SEQ ID NO: 12 - DNA, 171 bp, Streptococcus sp.
  source 1..171: mol_type = other DNA
gctaataccgcataagagtagatgttgcatgacatttgcttaaaaggtgcaattgcatcactaccagatggacctgcgttgtattagctagttggtggggtaacggctcaccaaggcgacgatacatagccgacctgagagggtgatcggccacactgggactgagacacg

SEQ ID NO: 13 - DNA, 1529 bp, unidentified
  source 1..1529: mol_type = other DNA
tattgagagtttgatcctggctcaggatgaacgctggcggcgtgcttaacacatgcaagtcgaacggaacacccttgaaggagttttcggacaactgagaggactgtttagtggcggacgggtgagtaacgcgtgagtaacctgccttggagtggggaataacacaaagaaatttgtgctaataccgcataatgcagctgagtcgcatggctctggctgccaaagatttatcgctctgagatggactcgcgtctgattagctagttggcggggtaacggcccaccaaggcgacgatcagtagccggactgagaggttggccggccacattgggactgagacacggcccagactcctacgggaggcagcagtggggaatattgggcaatgggcgcaagcctgacccagcaacgccgcgtgaaggaagaaggctttcgggttgtaaacttcttttctcagggacgaagcaagtgacggtacctgaggaataagccacggctaactacgtgccagcagccgcggtaatacgtaggtggcaagcgttatccggatttactgggtgtaaagggcgtgtaggcgggaaagcaagtcagatgtgaaaaccatgggctcaacctgtggcctgcatttgaaactgtttttcttgagtactggagaggcagacggaattcctagtgtagcggtgaaatgcgtagatattaggaggaacaccagtggcgaaggcggtctgctggacagcaactgacgctgaggcgcgaaagcgtggggagcaaacaggattagataccctggtagtccacgctgtaaacgatggatactaggtgtggggggactgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcccacggacagatgtagagatacatccttcccttcggggactgtggtgacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggtggtcaacagagggaagcaatgccgcgaggcggagcaaatccctaaaagccatcccagttcggatcgcaggctgcaacccgcctgcgtgaagttggaatcgctagtaatcgcggatcagcatgccgcggtgaatacgttcccgggccttgtacacaccgcccgtcacaccatgagagtcgggaacacccgaagtccgtagcctaaccgcaaggggggcgcggccgaaggtgggttcgataattggggtgaagtcgtaacaaggtagccgttcgagaacgagcggctggatcacctccttt

SEQ ID NO: 14 - DNA, 1529 bp, unidentified
  source 1..1529: mol_type = other DNA
ttcgagagtttgatcctggctcaggatgaacgctggcggcgtgcctaacacatgcaagtcgaacggggtcatgaattggaagtttacggatggaagagacatgacttagtggcggacgggtgagtaacgcgtgggtaacctgccgtatacagggggataacacttagaaataggtgctaacaccgcataagcgcacgggattgcatgatcttgtgtgaaaatctccggaggtatacgatgggcccgcgtctgattagccagttggcagggtaacggcctaccaaagcgacgatcagtagccggcctgagagggtggacggccacattgggactgagacacggcccaaactcctacgggaggcagcagtgggggatattgcacaatgggggaaaccctgatgcagcgacgccgcgtgagtgaagaagtatttcggtatgtaaagctctatcagcagggaagataatgacggtacctgagtaagaagccccggctaactacgtgccagcagccgcggtaatacgtagggggcaagcgttatccggatttactgggtgtaaagggagcgtagacggcagggcaagtctgatgtgaaagcccggggctcaaccccgggactgcattggaaactgtcaggctagagtgcaggagaggtgagcggaattcctagtgtagcggtgaaatgcgtagatattaggaggaacaccagtggcgaaggcggctcactggactgtaactgacgttgaggctcgaaagcgtggggagcaaacaggattagataccctggtagtccacgccgtaaacgatgaatactaggtgtcggtggttaaagaaccatcggtgccgcagcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccccctgaataaagggtaatgccgatagcccttcggggcaggggagacaggtggtgcatggttgtcgtcagctcgtgtcgtgagatgttgggttaagtcccgcaacgagcgcaacccctatccttagtagccagcaggtaaagctgggcactctagggagacagccggggataacccggaggaaggcggggatgacgtcaaatcatcatgccccttatgacttgggctacacacgtgctacaatggcgtgaacaaagtgaagcgagccagtgatggtaagcaaaacacagaaagcacgtctcagttcggattgtagtctgcaacccgactacatgaagctggaatcgctagtaatcgcggatcagaatgccgcggtgaatacgttcccgggtcttgtacacaccgcccgtcacaccatgggagtcggaaatgcccgaagccggtgaccgaaccgaaaggaaggagccgtcgaaggcaggatcgataactggggtgaagtcgtaacaaggtagccgtatcggaaggtgcggctggatcacctccttt

SEQ ID NO: 15 - DNA, 1533 bp, unidentified
  source 1..1533: mol_type = other DNA
tttatgagagtttgatcctggctcaggatgaacgctggcggcgtgcctaacacatgcaagtcgaacggggcgcgcaggctgaggcttttcggagcggaagcctgcgctgcccagtggcggacgggtgagtaacgcgtgggcaacctgccctgcactgggggacaacagccggaaacggctgctaataccgcatatgcccgcagccccgcatggggcggcgggggaagctccggcggtgcaggatgggcccgcgtctgattagctggttggcggggcaacggcccaccaaggcgacgatcagtagccggcctgagagggtgggcggccacattgggactgagacacggcccagactcctacgggaggcagcagtgggggatattgcacaatggggggaaccctgatgcagcgacgccgcgtgggcgatggagtgcttcggcatgtaaagccctatcagcagggaagaaaatgacggtacctgactaagaagccccggctaaatacgtgccagcagccgcggtaatacgtatggggcaagcgttatccggatttactgggtgtaaagggagcgtaggcggcctggcaagcctgatgtgaaataccggggcccaaccccggggctgcattgggaactgccaggctggagtgccggagaggcaggcggaattcctagtgtagcggtgaaatgcgtagatattaggaggaacaccagtggcgaaggcggcctgctggacggtgactgacgctgaggctcgaaagcgtggggagcaaacaggattagataccctggtagtccacgccgtaaacgatgaatactaggtgtcggcgggcaaagcccgccggtgccgcagcaaacgcagtaagtattccacctggggagtacgttcgcaagaatgaaactcaaaggaattgacggggacccgcacaagcggtggagcatgtggtttaattcgaagcaacgcgaagaaccttaccaggccttgacatcctgctgacggcatatgcaatgtatgttccctccggggcagcagagacaggtggtgcatggttgtcgtcagctcgtgtcgtgagatgttgggttaagtcccgcaacgagcgcaacccctgcccccagtagccagcgcgcaagggcgggcactctggggggactgccagggacaacctggaggaaggcggggatgacgtcaaatcatcatgccccttatggcctgggctacacacgtgctacaatggcgcccacaaagggaggcgaaatggcgacatggagcaaaccccagaaaaggcgccccagttcggattgcagcctgcaactcggctgcatgaagccggaatcgctagtaatcgcggatcagaatgccgcggtgaatacgttcccgggtcttgtacacaccgcccgtcacaccatgggagccgggaatgcccgaagtcagtggcccaaccgcaaggggggagctgccgaaggcaggcccggtgactggggtgaagtcgtaacaaggtagccgtatcggaaggtgcggctggatcacctccttt

SEQ ID NO: 16 - DNA, 1514 bp, unidentified
  source 1..1514: mol_type = other DNA
aattaagagtttgatcctggctcaggatgaacgctggcggcgtgcctaacacatgcaagtcgagcgagaagctgatgacagatacttcggttgaaggagtcagtggaaagcggcggacgggtgagtaacgcgtaggcaacctgccctttgcacagggatagccattggaaacgatgattaaaacctgataacaccatttggttacatgagcagatggtcaaagatttatcggcaaaggatgggcctgcgtctgattagctagttggtaaggtaacggcttaccaaggcgacgatcagtagccgacctgagagggtgaacggccacattggaactgagacacggtccaaactcctacgggaggcagcagtggggaatattgcacaatgggcgaaagcctgatgcagcaacgccgcgtgaaggaagaaggccttcgggtcgtaaacttctgtccttggggaagaagaactgacggtacccaaggaggaagccccggctaactacgtgccagcagccgcggtaatacgtagggggcaagcgttatccggaattattgggcgtaaagagtacgtaggtggcaacctaagcgcagggtttaaggcaatggctcaaccattgttcgccctgcgaactgggatgcttgagtgcaggagaggaaagcggaattcctagtgtagcggtgaaatgcgtagatattaggaggaacaccagtggcgaaggcggctttctggactgtaactgacactgaggtacgaaagcgtggggagcaaacaggattagataccctggtagtccacgccgtaaacgatgagcactaggtgtcggggtcgcaagacttcggtgccgtagttaacgcattaagtgctccgcctggggagtacgcacgcaagtgtgaaactcaaaggaattgacggggacccgcacaagcagcggagcatgtggtttaattcgaagcaacgcgaagaaccttaccaggacttgacatccctctgacagacccttaatcgggtttttctacggacagaggagacaggtggtgcatggttgtcgtcagctcgtgtcgtgagatgttgggttaagtcccgcaacgagcgcaacccttgccattagttgccagcagtaagatgggcactctagtgggactgccggggacaactcggaggaaggtggggatgacgtcaaatcatcatgccccttatgttctgggctacacacgtgctacaatggccggtacagaaagcagcgaagtcgtgaggcggagcaaatctcgaaaaccggtcccagttcggactgcaggctgcaactcgcctgcacgaagccggagttgctagtaatcgcggatcagaatgccgcggtgaatgcgttcccgggtcttgtacacaccgcccgtcacaccatggaagttgggggtgcccaaagccggcaaggaaatatgctgtctaaggcaaaaccaatgactggggtgaagtcgtaacaaggtagccgtatcggaaggtgcggctggatcacctccttt

SEQ ID NO: 17 - DNA, 1529 bp, unidentified
  source 1..1529: mol_type = other DNA
tattgagagtttgatcctggctcaggatgaacgctggcggcgtgcttaacacatgcaagtcgaacggaacacccttgaaggagttttcggacaactgagaggactgtttagtggcggacgggtgagtaacgcgtgagtaacctgccttggagtggggaataacacagagaaatttgtgctaataccgcataatgcagctgagtcgcatggctctggctgccaaagatttatcgctctgagatggactcgcgtctgattagctagttggcggggtaacggcccaccaaggcgacgatcagtagccggactgagaggttggccggccacattgggactgagacacggcccagactcctacgggaggcagcagtggggaatattgggcaatgggcgcaagcctgacccagcaacgccgcgtgaaggaagaaggctttcgggttgtaaacttcttttctcagggacgaagcaagtgacggtacctgaggaataagccacggctaactacgtgccagcagccgcggtaatacgtaggtggcaagcgttatccggatttactgggtgtaaagggcgtgtaggcgggaaagcaagtcagatgtgaaaaccatgggctcaacctgtggcctgcatttgaaactgtttttcttgagtactggagaggcagacggaattcctagtgtagcggtgaaatgcgtagatattaggaggaacaccagtggcgaaggcggtctgctggacagcaactgacgctgaggcgcgaaagcgtggggagcaaacaggattagataccctggtagtccacgctgtaaacgatggatactaggtgtggggggactgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcccacggacagatgtagagatacatccttcccttcggggactgtggtgacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggtggtcaacagagggaagcaatgccgcgaggcggagcaaatccctaaaagccatcccagttcggatcgcaggctgcaacccgcctgcgtgaagttggaatcgctagtaatcgcggatcagcatgccgcggtgaatacgttcccgggccttgtacacaccgcccgtcacaccatgagagtcgggaacacccgaagtccgtagcctaaccgcaaggggggcgcggccgaaggtgggttcgataattggggtgaagtcgtaacaaggtagccgttcgagaacgagcggctggatcacctccttt

SEQ ID NO: 18 - DNA, 1532 bp, unidentified
  source 1..1532: mol_type = other DNA
atgagagtttgatcctggctcaggatgaacgctggcggcgtgcttaacacatgcaagtcgaacgaagcactgaaagcagattacttcggtttgaagctttctttgactgagtggcggacgggtgagtaacgcgtgggtaacctgcctcatacagggggataacagttagaaatgactgctaataccgcataagcgcacggtttcgcatggaacagtgtgaaaaactccggtggtatgggatggacccgcgtctgattagctggttggcggggtaacggcccaccaaggcgacgatcagtagccggcctgagagggtgaacggccacattgggactgagacacggcccaaactcctacgggaggcagcagtggggaatattgcacaatgggggaaaccctgatgcagcgacgccgcgtgaaggaagaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtaggcggcgatgcaagtcagaagtgaaagcccggggctcaactccgggactgcttttgaaactgtgttgctagattgcaggagaggtaagtggaattcctagtgtagcggtgaaatgcgtagatattaggaggaacaccagtggcgaaggcggcttactggactgtaaatgacgctgaggctcgaaagcgtggggagcaaacaggattagataccctggtagtccacgccgtaaacgatgaatactaggtgtcagggagcaaggctctttggtgccgcagcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccggatgaccggaacttaaccgttccttcccttcggggcatccgagacaggtggtgcatggttgtcgtcagctcgtgtcgtgagatgttgggttaagtcccgcaacgagcgcaacccttatctttagtagccagcggaagaagccgggcactctagagagactgccagggataacctggaggaaggtggggatgacgtcaaatcatcatgccccttatgatttgggctacacacgtgctacaatggcgtaaacaaagggaagcgagcatgcgaatgtaagcaaatctcaaaaataacgtctcagttcggattgtagtctgcaactcgactacatgaagctggaatcgctagtaatcgcgaatcagcatgtcgcggtgaatacgttcccgggtcttgtacacaccgcccgtcacaccatgggagttggtaacgcccgaagccagtgacccaaccggaaacggagggagctgtcgaaggtgggaccgataactggggtgaagtcgtaacaaggtagccgtatcggaaggtgcggctggatcacctccttt

SEQ ID NO: 19 - DNA, 650 bp, unidentified; identical to SEQ ID NO: 1
  source 1..650: mol_type = other DNA

SEQ ID NO: 20 - DNA, 650 bp, unidentified
  source 1..650: mol_type = other DNA
agcggcgaacgggtgagtaacacgtaggtgatctgcccatcagacggggacaacgattggaaacgatcgctaataccggataggacgaaagtttaaagatgctcctggcatcactgatggatgagcctgcggcgcattagctagttggtggggtaaaggcctaccaaggcgacgatgcgtagccgacctgagagggtgaacggccacactgggactgagacacggcccagactcctacgggaggcagcagtagggaatcttcggcaatgggcgaaagcctgaccgagcaacgccgcgtgaatgaagaaggccttcgggttgtaaaattctgttataagggaagaaaggtgataggaggaaatgactatcaattgacggtaccttatgagaaagccacggctaactacgtgccagcagccgcggtaatacgtaggtggcaagcgttatccggaattattgggcgtaaagagcgcgcaggtggttaattaagtctgatgtgaaagcccacggcttaaccgtggagggtcattggaaactggttgacttgagtgcagaagagggaagtggaattccatgtgtagcggtgaaatgcgtagagatatggaggaacaccagtggcgaaggcggcttcctggtctgcaactgacact

SEQ ID NO: 21 - DNA, 621 bp, unidentified
  source 1..621: mol_type = other DNA
agcggcggacgggtgagtaacgcgtgggcaacctgccttacacagggggataacagttagaaatgactgctaataccgcataagaccgcagtaccgcatggtacagcggtaaaaactccggtggtgtaagatgggcccgcgtctgattaggtagttggcggggtaacggcccaccaagccgacgatcagtagccgacctgagagggtgaccggccacattgggactgagacacggcccagactcctacgggaggcagcagtggggaatattgcacaatgggggaaaccctgatgcagcgacgccgcgtgagcgatgaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggctgtgcaagccanatgtgaaagcccggggctcaaccccgggactgcatttggaactgtgtggctggagtgtcngagaggcaggcggaattcctagtgtancggtgaaatgcgtagatattangaggaacaccagtggcgaangcggcctgctgga

SEQ ID NO: 22 - DNA, 1271 bp, unidentified
  source 1..1271: mol_type = other DNA
tgacgaagcttgcttttgaattcttgtgtatcttagtggcggacgggtgagtaacgcgtgagcaacctgcctttcagagggggataacgtttggaaacgaacgctaataccgcatgagactacagtactacatggtacagtggccaaaggagcaatccgctgaaagatgggctcgcgtccgattagatagttggcggggtaacagcccaccaagtcgacgatcggtagccggactgagaggttgaacggccacattgggactgagacacggcccagactcctacgggaggcagcagtgagggatattggtcaatgggggaaaccctgaaccagcaacgccgcgtgagggaagacggttttcggattgtaaacctctgtcctctgtgaagataatgacggtagcagaggaggaagctccggctaactacgtgccagcagccgcggtaatacgtagggagcaagcgttgtccggatttactgggtgtaaagggtgcgtaggcggccttgcaagtcagaagtgaaatccatgggcttaacccgtgaactgcttttgaaactgtagggcttgagtgaagtagaggcaggcggaattcccggtgtagcggtgaaatgcgtagagatcgggaggaacaccagtggcgaaggcggccgagtggcggacgggtgagtaacgcgtgggtaacctgcctcatacagggggataacagttagaaatgactgctaataccgcataagcgcacggtctcgcatgggacagtgtgaaaaactccggtggtatgagatggacccgcgtctgattagctggttggtgaggtaagggctcaccaaggcgacgatcagtagccggcctgagagggtgaacggccacattgggactgagacacggcccaaactcctacgggaggcagcagtggggaatattgcacaatgggggaaaccctgatgcagcgacgccgcgtgaaggaagaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtaggcggttcagcaagtcagaagtgaaagcccggggctcaactccgggactgcttttgaaactgttgaactagattgcaggagaggtaagtggaattcctagtgtagcggtgaaatgcgtagatattaggaggaacaccagtggcgaaggcggcttactgg

SEQ ID NO: 23 - DNA, 621 bp, unidentified
  source 1..621: mol_type = other DNA
gagtggcggacgggtgagtaacgcgtgggtaacctgcctcatacagggggataacagttagaaatgactgctaataccgcataagcgcacggtctcgcatgggacagtgtgaaaaactccggtggtatgagatggacccgcgtctgattagctggttggtgaggtaagggctcaccaaggcgacgatcagtagccggcctgagagggtgaacggccacattgggactgagacacggcccaaactcctacgggaggcagcagtggggaatattgcacaatgggggaaaccctgatgcagcgacgccgcgtgaaggaagaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtaggcggttcagcaagtcagaagtgaaagcccggggctcaactccgggactgcttttgaaactgttgaactagattgcaggagaggtaagtggaattcctagtgtagcggtgaaatgcgtagatattaggaggaacaccagtggcgaaggcggcttactgg

SEQ ID NO: 24 - DNA, 621 bp, unidentified
  source 1..621: mol_type = other DNA
tcttagtggcggacgggtgagtaacgcgtggggaacctgccctgtacagggggataacacttanaaataggtgctaataccgcataagcgcacggtatcgcatgatacaatgtgaaaaactccggtggtacaggatggacccgcgtctgattagctggttggcggggtaacggcccaccaaggcgaccatcattagccggcctgagagggtgaacggacacattgggactganacacggcccaaactcctacgggaggcagcattggggaatattggacaatggggggaaccctgatccagcgacgccgcgtgagtgaagaagtatttcggtatgtaaagctctatcagcagggaagaaaatgacggaacctgactaagaagccccggctaactacgtgccagcagccgcggtaatacgtanggggcaagcgttatccggatttactgggtgtaaagggagcgtagacggtgcaacaagtctgatgtgaaaggtcngggcccaaccccgggactgcattggaaactgttgaactggaatacaggagangtaancggaattcctantgtancggtgaaatgcgtnnntattacgaggaacaccagtggcnaaggcggcttac

SEQ ID NO: 25 - DNA, 646 bp, unidentified
  source 1..646: mol_type = other DNA
gaacgggtgagtaacacgtaggtgatctgcccatcagacggggacaacgattggaaacgatcgctaataccggataggacgaaagtttaaagatgctcctggcatcactgatggatgagcctgcggcgcattagctagttggtggggtaaaggcctaccaaggcgacgatgcgtagccgacctgagagggtgaacggccacactgggactgagacacggcccagactcctacgggaggcagcagtagggaatcttcggcaatgggcgaaagcctgaccgagcaacgccgcgtgaatgaagaaggccttcgggttgtaaaattctgttataagggaagaaaggtgataggaggaaatgactatcaattgacggtaccttatgagaaagccacggctaactacgtgccagcagccgcggtaatacgtaggtggcaagcgttatccggaattattgggcgtaaagagcgcgcaggtggttaattaagtctgatgtgaaagcccacggcttaaccgtggagggtcattggaaactggttgacttgagtgcagaagagggaagtggaattccatgtgtagcggtgaaatgcgtagagatatggaggaacaccagtggcgaaggcggcttcctggtctgcaactgacactga

SEQ ID NO: 26 - DNA, 621 bp, unidentified
  source 1..621: mol_type = other DNA
tggcggacgggtgagtaacgcgtgggcaacctgccccacacagggggataacagccggaaacggctgctaataccgcataagcgcacagcttcgcatggagcggtgtgaaaagcctttgggcggtgtgggatgggcccgcgtctgattagctggttggcggggcagcggcccaccaaggcgacgatcagtagccggcctgagagggtggacggccacattgggactgagacacggcccagactcctgcgggaggcagcagtggggaatattgcacaatgggggaaaccctgatgcagcgacgccgcgtgagcgaagaagtatttcggtatgtaaagctctatcagcagggaagaaaatgacggtacctgagtaagaagctccggctaaatacgtgccagcagccgcggtaatacgtatggagcaagcgttatccggatttactgggtgtaaagggagcgcaggcggccaggcaagtctgatgtgaaataccggggctcaaccccggaactgcattggaaactgtcaggctggagtgtcggagaggcaggcggaattcctggtgtagcggtgaaatgcgtagatatcaggaggaacaccagtggcgaaggcggcctgctgga

SEQ ID NO: 27 - DNA, 620 bp, unidentified
  source 1..620: mol_type = other DNA
ggacgggtgagtaacgcgtaggcaacctgcccttagcacagggatagccattggaaacgatgattaaaacctgataacaccatttggttacatgagcagatggtcaaagatttatcggcaaaggatgggcctgcgtctgattagctagttggtaaggtaacggcttaccaaggcgacgatcagtagccgacctgagagggtgaacggccacattggaactgagacacggtccaaactcctacgggaggcagcagtggggaatattgcacaatgggcgaaagcctgatgcagcaacgccgcgtgaaggaagaaggccttcgggtcgtaaacttctgtccttggggaagaagaactgacggtacccaaggaggaagccccggctaactacgtgccagcagccgcggtaatacgtagggggcaagcgttatccggaattattgggcgtaaagagtacgtaggtggcaacctaagcgcagggtttaaggcaatggctcaaccattgttcgccctgcgaactgggatgcttgagtgcaggagaggaaagcggaattcctagtgtagcggtgaaatgcgtagatattaggaggaacaccagtggcgaaggcggctttctggactgt

SEQ ID NO: 28 - DNA, 645 bp, unidentified
  source 1..645: mol_type = other DNA
tcggttgaaggagtcagtggaaagcggcggacgggtgagtaacgcgtaggcaacctgccctttgcacagggatagccattggaaacgatgattaaaacctgataacaccatttggttacatgagcagatggtcaaagatttatcggcaaaggatgggcctgcgtctgattagctagttggtaaggtaacggcttaccaaggcgacgatcagtagccgacctgagagggtgaacggccacattggaactgagacacggtccaaactcctacgggaggcagcagtggggaatattgcacaatgggcgaaagcctgatgcagcaacgccgcgtgaaggaagaaggccttcgggtcgtaaacttctgtccttggggaagaagaactgacggtacccaaggaggaagccccggctaactacgtgccagcagccgcggtaatacgtagggggcaagcgttatccggaattattgggcgtaaagagtacgtaggtggcaacctaagcgcagggtttaaggcaatggctcaaccattgttcgccctgcgaactgggatgcttgagtgcaggagaggaaagcggaattcctagtgtagcggtgaaatgcgtagatattaggaggaacaccagtggcgaaggcggctttctggac

SEQ ID NO: 29 - DNA, 636 bp, unidentified
  source 1..636: mol_type = other DNA
attcatattgactgagtggcggacgggtgagtaacgcgtgggtaacctgcctcatacagggggataacagttagaaatgactgctaataccgcataagcgcacagaatcgcatgattcggtgtgaaaaactccggtggtatgagatggacccgcgtctgattagctagttggtggggtaaaggcctaccaaggcgacgatcagtagccgacctgagagggtgatcggccacattgggactgagacacggcccaaactcctacgggaggcagcagtggggaatattgcacaatgggggaaaccctgatgcagcaacgccgcgtgagtgaagaagtatttcgatatgtaaagctctatcagcagggaagaagatgacggtacctgagtaagaagccccggctaactacgtgccagcagccgcggtaatacgtagggggcaagcgttatccggatttactgggtgtaaagggagcgtagacggcatagcaagtctgaagtgaaagcccaaggctcaaccatgggactgctttggaaactgttaagctagagtgctggagaggtaagcggaattcctagtgtagcggtgaaatgcgtagatattaggaagaacaccagtggcgaanncggcttactggnc

SEQ ID NO: 30 - DNA, 625 bp, unidentified
  source 1..625: mol_type = other DNA
gactgagtggcggacgggtgagtaacgcgtgggcaacctgccccacacagggggataacagccggaaacggctgctaataccgcataagcgcacagcttcgcatggagcggtgtgaaaagcctttgggcggtgtgggatgggcccgcgtctgattagctggttggcggggcagcggcccaccaaggcgacgatcagtagccggcctgagagggtggacggccacattgggactgagacacggcccagactcctgcgggaggcagcagtggggaatattgcacaatgggggaaaccctgatgcagcgacgccgcgtgagcgaagaagtatttcggtatgtaaagctctatcagcagggaagaaaatgacggtacctgagtaagaagctccggctaaatacgtgccagcagccgcggtaatacgtatggagcaagcgttatccggatttactgggtgtaaagggagcgcaggcggccaggcaagtctgatgtgaaataccggggctcaaccccggaactgcattggaaactgtcaggctggagtgtcggagaggcaggcggaattcctggtgtagcggtgaaatgcgtagatatcaggaggaacaccagtggcgaaggcggcctgct

SEQ ID NO: 31 - DNA, 621 bp, unidentified
  source 1..621: mol_type = other DNA
agcggcggacgggtgagtaacgcgtgggcaacctgccttacacagggggataacagttagaaatgactgctaataccgcataagaccgcagtaccgcatggtacagcggtaaaaactccggtggtgtaagatgggcccgcgtctgattaggtagttggcggggtaacggcccaccaagccgacgatcagtagccgacctgagagggtgaccggccacattgggactgagacacggcccagactcctacgggaggcagcagtggggaatattgcacaatgggggaaaccctgatgcagcgacgccgcgtgagcgatgaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtanacggctgtgcaagccagatgtgaaagcccggggctcaaccccgggactgcatttggaactgtgtggctggagtgtcngagaggcaggcggaattcctagtgtancggtgaaatgcgtanatattaggaggaacaccagtggcgaaggcggcctgctgga

(19) United States
(12) Patent Application Publication
(10) Pub. No.: US 2026/0014213 A1
(43) Pub. Date: Jan. 15, 2026

(54) BACTERIAL STRAINS FROM CLOSTRIDIA OR BACILLI AND METHODS OF TREATING OBESITY, METABOLIC SYNDROME, DIABETES AND INFLAMMATORY BOWEL DISEASE

(71) Applicant: UNIVERSITY OF UTAH RESEARCH FOUNDATION, Salt Lake City, UT (US)

(72) Inventors: June L. ROUND, Salt Lake City, UT (US); Kendra KLAG, East Salt Lake City, UT (US)

(21) Appl. No.: 19/111,926

(22) PCT Filed: Sep. 14, 2023
(86) PCT No.: PCT/US2023/074202

§ 371 Date: Mar. 14, 2025

Related U.S. Application Data
(60) Provisional application No. 63/407,014, filed on Sep. 15, 2022.

(51) Int. Cl. A61K 35/742; A61P 3/04; A61P 3/06; A61P 3/10

(52) U.S. Cl. CPC A61K 35/742; A61P 3/04; A61P 3/06; A61P 3/10

(57) ABSTRACT

Disclosed herein, are compositions comprising bacterial strains from Clostridia or Bacilli, and methods of treating obesity, metabolic syndrome, inflammatory bowel disease, as well as reducing weight gain, inhibiting lipid absorption in the small intestine and downregulating CD36 in the liver or small intestine by administering the compositions to a subject.

### CROSS REFERENCE TO RELATED APPLICATIONS

This application claims the benefit of U.S. Provisional Application No. 63/407,014, filed Sep. 15, 2022. The content of this earlier filed application is hereby incorporated by reference herein in their entirety.

### INCORPORATION OF THE SEQUENCE LISTING

The present application contains a sequence listing that is submitted concurrent with the filing of this application, containing the file name “21101_0443P1_SL.xml” which is 53,248 bytes in size, created on Sep. 7, 2023, and is herein incorporated by reference in its entirety.

### BACKGROUND

The gut microbiota is an important factor regulating mammalian metabolism. The gut microbial metagenome encodes a diverse array of functions that complement host genes involved in metabolism. Maintenance of the diversity and collective functional capacity of the microbiota may be important to the promotion of optimal metabolic health throughout life. The microbiota influences host metabolism and obesity, yet organisms that protect from obesity remain unknown.

The prevalence of obesity has grown in an alarming rate in the past 20 years. Based on an estimate in 2004, in the U.S. alone, 66.3% of adults are either overweight or obese, and 32.2% of adults are classified as obese (Cynthia L. Ogden et al., JAMA 2006 Apr. 5; 295:1549-1555). Both genetic and environmental factors have been shown to cause positive energy balance and obesity. Obesity by itself is only a part of problems. Many other chronic diseases such as type 2 diabetes, certain cancers and cardiovascular diseases are common co-morbidities of obesity. Collectively, obesity associated medical issues put a tremendous amounts of pressure on health care systems in many countries.

Drug treatments for obesity are available but not very effective and with undesirable side-effects. Still more drugs are under development to improve the safety, efficacy of the medications and convenience to use them by patients. To date, the anti-obesity treatments available are designed to alter the internal metabolism of patients. Most of these drugs are required to be absorbed and delivered to target organs through blood stream for their efficacy. Safety concerns of such a treatment strategy cannot be ignored.

Treatment strategies of obesity and type 2 diabetes focusing on targets outside of human tissues is greatly desirable because the active agents are not required to enter the body, and the safety of the treatments can be improved significantly.

Recent research has shown that gut bacteria play a role in the development of obesity and related metabolic disorders such as diabetes (Kristina Harris, et al., Journal of Obesity 2012; 2012:879151; doi: 10.1155/2012/879151). Human beings are super-organisms with a body composed of millions of human cells while many more bacteria live, e.g., in the colon. It has been estimated that more than 10¹³ to 10¹⁴ bacteria are alive in a healthy human intestine. Intestinal bacteria can be separated into two major divisions, Firmicutes and Bacteriodetes (Steven R. Gill, et al., Science 2006 Jun. 2; 312:1355-1359; Peter J. Turnbaugh, et al., Nature 2006 Dec. 21; 444:1027-131). Together, they represent at least 90% of total bacterial population in the gut. The presence of the gut bacteria is a part of normal human physiology and is important for the development of gut functions (Hooper L V et al., Science. 2001 Feb. 2; 291 (5505):881-4; Stappenbeck T S, et al., Proc Natl Acad Sci USA. 2002 Nov. 26; 99(24): 15451-5), maturation of the immune system (Mazmanian S K, et al., Cell. 2005 Jul. 15; 122 (1): 107-18), harvesting energy from dietary carbohydrates (Peter J. Turnbaugh, et al., Nature 2006 Dec. 21; 444:1027-131), harvesting essential vitamins (Backhed F, et al., Science. 2005 Mar. 25; 307 (5717): 1915-20) and metabolizing environmental chemicals in the gut (Nicholson J K, et al., Nat Rev Microbiol. 2005 May; 3(5): 431-8). Recent studies further suggested that gut bacteria may be involved in fat storage (Backhed F, et al., Proc Natl Acad Sci USA. 2004 Nov. 2; 101 (44): 15718-23).

It is know that the modulation of the gut microbiota during infancy can prospectively have a great influence in the future health status of the bodies, in particular the development of obesity later in life. Such modulation can be achieved by introducing probiotics in the food consumed.

WO 2006/019222 discloses Lactobacillus rhamnosus strain PL60 KCCM-10654P with a body-fat reducing activity that overproduces t10c12-octadecadienoic acid. U.S. Pat. No. 7,001,756 and CN1670183 provide an isolated microorganism strain Lactobacillus rhamnosus GM-020 which is found to be effective in treating obesity. WO 2009/0218424 describes a composition comprising Lactobacillus rhamnosus strain CGMCC 1.3724 or NCC4007 which is useful for supporting weight loss or weight management.

WO 2009/024429 describes a similar composition comprising Lactobacillus rhamnosus strain CGMCC 1.3724 or NCC4007 for the use in treating or preventing metabolic disorders. The composition was shown to modify the amount of Proteobacteria in the gut. Optimum results were achieved when the ratio of Proteobacteria to Bacteriodetes was reduced. At the same time, the ratio of Proteobacteria to Firmicutes and/or the ratio of Bacteriodetes to Firmicutes may be increased.

Another approach is to introduce specific nutrients that influence the development of the gut microbiota. Such nutrients can be vitamins, particular proteins, specific fats, or carbohydrates. Some prebiotic oligosaccharides have been described to influence the microbiota of the gut and further have been associated with weight loss or reduction of risk of obesity.

WO2011096808 assigned to Friesland Bands B V, described that sialyl-oligosaccharides in infant formula can enhance the amount of Bacteroides ssp. in the gastrointestinal tract and therefore reduce the risk of development of overweight or obesity. WO2009082214 assigned to N. V. Nutricia, describes that a combination of at least 2 non digestible carbohydrates (prebiotics) can modulate the microbiota in infants, especially decreasing the ratio of Firmicutes/Bacteroidetes and/or Clostridium/Bacteroidetes. It is reported that such modulation can act for the prevention of obesity or adiposity. WO2012024638 assigned to New York University, Dow Global technologies LLC. Nondorf, Laura and Cho Ilseung, describes the down-modulation of Firmicutes and/or Bacteroidetes in the ileal microbiota of mammals. Such modulation can be achieved by the ingestion of saccharides and lead to the treatment or prevention of obesity.

EP2143341A1, assigned to Nestec S A, describes the reduction of obesity later in life by the use of specific oligosaccharide mixtures in nutritional compositions for infants and young children.

However further effectors and modulator of the microbiota still remain to be found. It is a problem of the present invention to provide additional or alternative means for modulating the gut microbiota in order to modulate the accumulation of fat mass, modulate the adiposity and/or reduce the risk of obesity.

It is a problem of the present invention to provide additional or alternative ways of modulating the gut microbiota in order to modulate the accumulation of fat mass, modulate the adiposity and/or reduce the risk of obesity in a subject.

It is a problem of the present invention to provide additional or alternative solutions for re-establishing normal gut microbiota in population affected by suboptimal profile and/or unbalance of gut microbiota in a subject. It is a problem to effect such normalization of microbiota in a general or specific manner (specific to certain microorganisms of the gut flora) in a subject. It is a problem to effect such normalization in a way able to ultimately modulate the accumulation of fat mass, modulate the adiposity and/or reduce the risk of obesity in a subject.

It is a problem of the invention to ultimately help establishing a normal BMI (body Mass Index) in population at risk of having BMI above normality later in life (e.g. over-weight or obesity) in a subject.

### SUMMARY

Disclosed herein are consortia of bacteria. Disclosed herein are Clostridia consortia. Disclosed herein are Bacilli consortia.

Disclosed herein are compositions comprising bacterial strains from Clostridia.

Disclosed herein are compositions comprising a Clostridia consortium.

Disclosed herein are compositions comprising bacterial strains from Bacilli.

Disclosed herein are compositions comprising a Bacilli consortium.

Disclosed herein are compositions comprising bacterial strains from Clostridia or Bacilli, and a carrier.

Disclosed herein are a consortium of bacteria comprising two or more bacteria with a 16S nucleic acid sequence that is at least 97% identical to any of SEQ ID NOs: 1-31.

Disclosed herein are consortium of bacteria comprising two or more bacteria with a 16S nucleic acid sequence that is at least 97% identical to any of SEQ ID NOs: 1-31, wherein the consortium suppresses expression of lipid adsorption genes within intestinal epithelia in a subject compared to a subject where the consortium has not been administered.

Disclosed herein are methods of altering relative abundance of microbiota in a subject, the methods comprising administering to the subject an effective dose of any of the compositions described herein, thereby altering the relative abundance of microbiota in the subject.

Disclosed herein are methods of treating a subject with obesity. Disclosed herein are methods of treating a subject with obesity in a subject, the methods comprising administering to the subject an effective dose of any of the compositions described herein, thereby treating obesity in the subject. In some aspects, the relative abundance of spore-forming microbiota is increased in the subject compared to the relative abundance prior to administration.

Disclosed herein are methods of treating a subject with metabolic syndrome the methods comprising administering to the subject an effective dose of any of the compositions described herein, thereby treating metabolic syndrome in the subject. In some aspects, the relative abundance of spore-forming microbiota is increased in the subject compared to the relative abundance prior to administration.

Disclosed herein are methods of treating a subject with inflammatory bowel disease the methods comprising administering to the subject an effective dose of any of the compositions described herein, thereby treating inflammatory bowel disease in the subject. In some aspects, the relative abundance of spore-forming microbiota is increased in the subject compared to the relative abundance prior to administration.

Disclosed herein are methods of reducing weight gain in a subject the methods comprising administering to the subject an effective dose of any of the compositions described herein, thereby treating weight gain in the subject. In some aspects, the relative abundance of spore-forming microbiota is increased in the subject compared to the relative abundance prior to administration.

Disclosed herein are methods of inhibiting lipid absorption in a subject's small intestine the methods comprising administering to the subject an effective dose of any of the compositions described herein, thereby inhibiting lipid absorption in the subject's small intestine. In some aspects, the relative abundance of spore-forming microbiota is increased in the subject compared to the relative abundance prior to administration.

Disclosed herein are methods of downregulating CD36 in a subject's liver or small intestine the methods comprising administering to the subject an effective dose of any of the compositions described herein, thereby downregulating CD36 in the subject's liver or small intestine. In some aspects, the relative abundance of spore-forming microbiota is increased in the subject compared to the relative abundance prior to administration.

### BRIEF DESCRIPTION OF THE DRAWINGS

The accompanying figures, which are incorporated in and constitute a part of this specification, illustrate several aspects and together with the description serve to explain the principles of the invention.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

FIGS. 1A-D show the colonization of germ free (GF) mice with different bacteria from the spore-forming (SF) community modulates metabolism. Five week old male B6 WT GF mice were gavaged with 200 μL of pure culture, the SF community, or a community of 6 different cultured bacteria pooled together (referred to herein as “Cultured”; SEQ ID NOs: 7-12 in Table 2) in a sterile hood. Mice labeled “Turicibacter bubble” were colonized with Turicibacter since birth rather than at weaning. Percent fat (FIG. 1A), triglycerides (FIG. 1B), blood glucose (FIG. 1C) and insulin (FIG. 1D) were assessed.

FIGS. 2A-F show that Anaerovorax (SEQ ID NO: 3) lowers serum triglycerides on a High Fat Diet (HFD). Five week old male B6 SPF mice were put on a HFD (45% fat) at weaning and gavaged with 200 μL of culture (1×10⁴ to 1×10⁶ CFUs) five days/week or with vehicle control (reduced PBS with 0.1% L-Cystine). Mice were weighed weekly (FIG. 2C) and at 8 weeks of HFD treatment body fat was measured by NMR (FIG. 2A), mice were then fasted overnight and, after fasting blood glucose (FIG. 2D) was measured and serum taken for insulin (FIG. 2E) and triglyceride (FIG. 2B) measurements. HOMA-IR is a measure of insulin resistance (FIG. 2F).

FIGS. 3A-G show that Turicibacter (SEQ ID NO: 1) conveys metabolic protection on a High Fat Diet (HFD). Five week old male B6 SPF mice were put on a HFD (45% fat) at weaning and gavaged with 200 μL of culture (2×10⁵ CFUs) five days/week or with vehicle control (reduced PBS with 0.1% L-Cystine). Mice were weighed weekly (FIG. 3B) and at 8 weeks of HFD treatment body fat (FIG. 3A) was measured by NMR, then fasted overnight and blood measured for glucose (FIG. 3F) and serum taken for insulin (FIG. 3C) and triglyceride (FIG. 3E) measurements. HOMA-IR is a measure of insulin resistance (FIG. 3G). iWAT mass was also measured (FIG. 3D).

FIGS. 4A-F show that the SF consortium (comprising SEQ ID NOs: 1-31) protects mice from a model of inflammatory bowel disease. Six week old WT B6 Specific Pathogen Free mice were pretreated for 7 days with 200 μL/mouse/day of SF consortium and then treated with the SF consortium every other day for the duration. After 7 days of receiving the SF consortium, mice were given water containing 2.5% DSS for 5 days, followed by water for 10 days, followed by 2.5% dextran sulfate sodium (DSS) for 5 days, and followed by water for 10 days. Mice receiving the SF consortium were significantly protected from DSS induced intestinal inflammation (colon length, FIG. 4A; crypt loss, FIG. 4B; percent weight, FIG. 4C; crypt severity FIG. 4D; inflammation, FIG. 4E; and histology score, FIG. 4F).

### DETAILED DESCRIPTION

The present disclosure can be understood more readily by reference to the following detailed description of the invention, the figures and the examples included herein.

Before the present methods and compositions are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### DEFINITIONS

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile, cerebral spinal fluid) that contains cells or cell components. In some aspects, the sample can be taken from the brain, spinal cord, cerebral spinal fluid or blood.

As used herein, the term “subject” refers to the target of administration, e.g., a human. Thus the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment for altering relative abundance of spore-forming microbiota, such as, for example, prior to the administering step. In some aspects of the disclosed methods, the “patient” has been diagnosed with a need for treatment for metabolic disease, type II diabetes, obesity, or an inflammatory bowel disease, such as, for example, prior to the administering step.

As used herein, the term “normal” refers to an individual, a sample or a subject that does not have a disease (e.g., metabolic disease, type II diabetes, obesity, or an inflammatory bowel disease) or does not have an increased susceptibility of developing a disease (e.g., metabolic disease, type II diabetes, obesity, or an inflammatory bowel disease).

As used herein, the term “susceptibility” refers to the likelihood of a subject being clinically diagnosed with a disease. For example, a human subject with an increased susceptibility a for metabolic disease, type II diabetes, obesity, or an inflammatory bowel disease can refer to a human subject with an increased likelihood of a subject being clinically diagnosed with a metabolic disease, type II diabetes, obesity, or an inflammatory bowel disease.

As used herein, the term “comprising” can include the aspects “consisting of” and “consisting essentially of.”

As used herein, a “control” is a sample from either a normal subject or from tissue from a normal subject that does not have a metabolic disease, type II diabetes, obesity, or an inflammatory bowel disease.

As used herein, “over-expression” means expression greater than the expression detected in a normal sample. For example, a nucleic acid that is over-expressed may be expressed about 1 standard deviation above normal, or about 2 standard deviations above normal, or about 3 standard deviations above the normal level of expression. Therefore, a nucleic acid that is expressed about 3 standard deviations above a control level of expression is a nucleic acid that is over-expressed.

As used herein, “treat” is meant to mean administer a compound or composition of the invention to a subject, such as a human or other mammal (for example, an animal model), that has a metabolic disease, type II diabetes, obesity, or an inflammatory bowel disease, in order to prevent or delay a worsening of the effects of the disease or condition, or to partially or fully reverse the effects or symptoms of the disease.

As used herein, “prevent” is meant to mean minimize the chance that a subject who has an increased susceptibility for developing a disease (e.g., metabolic disease, type II diabetes, obesity, or an inflammatory bowel disease or will develop a metabolic disease, type II diabetes, obesity, or an inflammatory bowel disease) of actually developing the disease.

As used herein, the term “reference,” “reference expression,” “reference sample,” “reference value,” “control,” “control sample” and the like, when used in the context of a sample or expression level of one or more microbes refers to a reference standard wherein the reference is expressed at a constant level among different (i.e., not the same tissue, but multiple tissues) tissues, and is unaffected by the experimental conditions, and is indicative of the level in a sample of a predetermined disease status (e.g., not suffering from a metabolic disease, type II diabetes, obesity, or an inflammatory bowel disease). The reference value can be a predetermined standard value or a range of predetermined standard values, representing no illness, or a predetermined type or severity of illness.

As used herein, the term “spore-forming microbiota” or “spore-forming bacteria” or “spore-forming microbes” is meant to mean spores of bacterial species that are dormant bodies that carry the genetic material as found in the vegetative form, and do not have an active metabolism. In some aspects, spore-forming bacteria can include Bacillus and Clostridium species. In some aspects, spore-forming bacteria can be aerobic bacterial species and anaerobic bacterial species. In some aspects, the spore-forming bacteria described herein are capable of growing in both a vegetative and spore state when administered to a subject.

As used herein, the term “probiotic” means microbial cell preparations or components or metabolites of microbial cells with a beneficial effect on the health or well-being of the host (Salminen, S. et al. (1999); Probiotics: how should they be defined, Trends Food Sci. Technol., 10 107-10). The definition of probiotic is generally admitted and in line with the WHO definition. The probiotic can comprise a unique strain of microorganism, a mix of various strains and/or a mix of various bacterial species and genera. In case of mixtures, the singular term “probiotic” can still be used to designate the probiotic mixture or preparation. For the purpose of the present invention, spore-forming microbiota or spore-forming bacteria or spore-forming microbes are considered as probiotics.

“Prebiotic” generally means a non-digestible food ingredient that beneficially affects the host by selectively stimulating the growth and/or activity of micro-organisms present in the gut of the host, and thus attempts to improve host health (Gibson and Roberfroid “Dietary Modulation of the Human Colonic Microbiota: Introducing the Concept of Prebiotics”, J. Nutr. 1995: 125(6):1401-1412). “Prebiotics” alternatively means selectively fermented ingredients that allow specific changes, both in the composition and/or activity in the gastrointestinal microflora, that confer benefits upon the host well-being and health (Roberfroid M. “Prebiotics: the concept revisited”, J. Nutr. 2007:37 (3): 830S-837S). The percentages are by weight unless otherwise stated. The expressions “weight %” and “wt %” are synonymous. They refer to quantities expressed in percent on a dry weight basis.

### COMPOSITIONS

The present disclosure is directed to compositions containing and methods of using bacterial isolates and communities. In particular, the present disclosure is directed to a composition containing one or more bacterial microorganisms from the bacterial consortia as disclosed herein, particularly in Table 1, Table 2, Table 3 or mixtures thereof. In a preferred embodiment, the composition will include two or more strains of bacterium having a 16S rDNA sequence comprising SEQ ID NOs: 1, 2, 3, 4, 5, 6, 7, or 8. In an aspect, disclosed are compositions comprising two or more strains of bacterium having a 16S rDNA sequence 97% identical to SEQ ID NOs: 1, 2, 3, 4, 5, 6, 7, or 8. The bacterial microorganisms can be characterized by an identifying 16S ribosomal gene sequence corresponding to, and at and least 97% identical to one or more of SEQ ID NOs 1-8. In some aspects, the composition will include one or more strains of bacterium having a 16S rDNA sequence comprising one or more of SEQ ID NOs: 1-31. In some aspects, the composition will include two or more strains of bacterium having a 16S rDNA sequence comprising one or more of SEQ ID NOs: 1-31. In some aspect, the bacterial microorganisms can be characterized by an identifying 16S ribosomal gene sequence corresponding to, and at and least 97% identical to one or more of SEQ ID NOs 1-31.

Also disclosed herein are compositions comprising bacterial strains from Clostridia or Bacilli, and a carrier. Further disclosed herein are compositions comprising bacterial strains from Clostridia, Bacilli, or a combination thereof, and a carrier. In some aspects, the bacterial strains from Clostridia or Bacilli include an rRNA 16S having a nucleotide sequence greater than 700 nucleotides in length, wherein the nucleotide sequence is at least 97% identical to one of SEQ ID NOs: 1, 2, 3, 4, 5, 6, 7 or 8. In some aspects, the compositions disclosed herein can further comprise one or more bacterial strains selected from SEQ ID NOs: 9, 10, 11, 12, 13, 14, 15, 16, 17, 18 or 19. In some aspects, the compositions disclosed herein can further comprise Akkermansia muciniphila.

Also disclosed herein are compositions comprising bacterial strains from Clostridia, Bacilli, or a combination thereof, and a carrier. In some aspects, the bacterial strains from Clostridia or Bacilli include an rRNA 16S having a nucleotide sequence greater than 700 nucleotides in length, wherein the nucleotide sequence is at least 97% identical to one of SEQ ID NOs: 1-31.

Also disclosed herein are compositions consisting of bacterial strains from Clostridia or Bacilli, and a carrier. Further disclosed herein are compositions consisting of bacterial strains from Clostridia, Bacilli, or a combination thereof, and a carrier. In some aspects, the bacterial strains from Clostridia or Bacilli include an rRNA 16S having a nucleotide sequence greater than 700 nucleotides in length, wherein the nucleotide sequence is at least 97% identical to one of SEQ ID NOs: 1, 2, 3, 4, 5, 6, 7 or 8. In some aspects, the compositions disclosed herein can further consist of one or more bacterial strains selected from SEQ ID NOs: 9, 10, 11, 12, 13, 14, 15, 16, 17, 18 or 19. In some aspects, the compositions disclosed herein can further consist of Akkermansia muciniphila.

Also disclosed herein are compositions consisting of bacterial strains from Clostridia, Bacilli, or a combination thereof, and a carrier. In some aspects, the bacterial strains from Clostridia or Bacilli include an rRNA 16S having a nucleotide sequence greater than 700 nucleotides in length, wherein the nucleotide sequence is at least 97% identical to one of SEQ ID NOs: 1-31.

Disclosed herein are newly identified bacterium. It was found that the bacterium having a 16S rDNA sequence comprising SEQ ID NOs: 1, 2, 3, 4, 5, and 6, belong to the class Clostridia. In some aspects, the compositions described herein comprise at least one bacterium, wherein the bacteria is a Clostridia sp. It was found that the bacterium having a 16S rDNA sequence comprising SEQ ID NOs: 7 and 8, belong to the class Bacilli. In some aspects, the compositions described herein comprise at least one bacterium, wherein the bacteria is a Bacilli sp.

Disclosed herein compositions for reducing adiposity in a subject, reducing weight gain and/or fat accumulation in a subject, lowering body fat percentage and/or reducing visceral adipose tissue (VAT) mass in a subject, decreasing blood glucose levels and/or reducing insulin resistance in a subject, inhibiting lipid absorption in a subject's small intestine, downregulating CD36 in a subject's liver or small intestine, reducing serum triglycerides and suppressing expression of lipid absorption genes within intestinal epithelia in a subject. In some aspects, a combination of any two or more of the bacterial strains of Table 1 can be used in a composition. In some aspects, the various bacteria in the composition can be identified by their 16S ribosomal gene sequences. In some aspects, the compositions can include up to eight of the bacterial strains listed in Table 1, Table 2, Table 3 or a mixture thereof. In some aspects, the compositions can include two or more of the bacterial strains listed in Table 1, Table 2, Table 3 or a mixture thereof. In some aspects, the compositions can include three or more of the bacterial strains listed in Table 1, Table 2, Table 3 or a mixture thereof. In some aspects, the compositions can include four or more of the bacterial strains listed in Table 1, Table 2, Table 3 or a mixture thereof. In some aspects, the compositions can include five or more of the bacterial strains listed in Table 1, Table 2, Table 3 or a mixture thereof. In some aspects, the compositions can include six or more of the bacterial strains listed in Table 1, Table 2, Table 3 or a mixture thereof. In some aspects, the compositions can include seven or more of the bacterial strains listed in Table 1, Table 2, Table 3 or a mixture thereof. In some aspects, the compositions can include eight or more of the bacterial strains listed in Table 1, Table 2, Table 3 or a mixture thereof. In some aspects, the compositions can include nine or more of the bacterial strains listed in Table 1, Table 2, Table 3 or a mixture thereof. In some aspects, the compositions can include ten or more of the bacterial strains listed in Table 1, Table 2, Table 3 or a mixture thereof. In some aspects, the compositions can include eleven or more of the bacterial strains listed in Table 1, Table 2, Table 3 or a mixture thereof. In some aspects, the compositions can include twelve or more of the bacterial strains listed in Table 1, Table 2, Table 3 or a mixture thereof. In some aspects, the compositions can include thirteen or more of the bacterial strains listed in Table 1, Table 2, Table 3 or a mixture thereof. In some aspects, the compositions can include fourteen or more of the bacterial strains listed in Table 1, Table 2, Table 3 or a mixture thereof. In some aspects, the compositions can include fifteen or more of the bacterial strains listed in Table 1, Table 2, Table 3 or a mixture thereof. In some aspects, the compositions can include sixteen or more of the bacterial strains listed in Table 1, Table 2, Table 3 or a mixture thereof. In some aspects, the compositions can include seventeen or more of the bacterial strains listed in Table 1, Table 2, Table 3 or a mixture thereof. In some aspects, the compositions can include eighteen or more of the bacterial strains listed in Table 1, Table 2, Table 3 or a mixture thereof. In some aspects, the compositions can include nineteen or more of the bacterial strains listed in Table 1, Table 2, Table 3 or a mixture thereof. In some aspects, the compositions can include twenty or more of the bacterial strains listed in Table 1, Table 2, Table 3 or a mixture thereof. In some aspects, the compositions can include twenty-one or more of the bacterial strains listed in Table 1, Table 2, Table 3 or a mixture thereof. In some aspects, the compositions can include twenty-two or more of the bacterial strains listed in Table 1, Table 2, Table 3 or a mixture thereof. In some aspects, the compositions can include twenty-three or more of the bacterial strains listed in Table 1, Table 2, Table 3 or a mixture thereof. In some aspects, the compositions can include twenty-four or more of the bacterial strains listed in Table 1, Table 2, Table 3 or a mixture thereof. In some aspects, the compositions can include twenty-five or more of the bacterial strains listed in Table 1, Table 2, Table 3 or a mixture thereof. In some aspects, the compositions can include twenty-six or more of the bacterial strains listed in Table 1, Table 2, Table 3 or a mixture thereof. In some aspects, the compositions can include twenty-seven or more of the bacterial strains listed in Table 1, Table 2, Table 3 or a mixture thereof. In some aspects, the compositions can include twenty-eight or more of the bacterial strains listed in Table 1, Table 2, Table 3 or a mixture thereof. In some aspects, the compositions can include twenty-nine or more of the bacterial strains listed in Table 1, Table 2, Table 3 or a mixture thereof. In some aspects, the compositions can include thirty or more of the bacterial strains listed in Table 1, Table 2, Table 3 or a mixture thereof. In some aspects, the compositions can include thirty-one or more of the bacterial strains listed in Table 1, Table 2, Table 3 or a mixture thereof. In some aspects, the compositions can include one or more of the bacterial strains listed in Table 1, Table 2, Table 3 or a mixture thereof.

The present disclosure is directed to compositions containing and methods of using bacterial isolates and communities. Disclosed herein are spore-forming bacteria. Disclosed herein are bacterial strains from Clostridia or Bacilli. Disclosed herein are bacterial strains from Clostridia or Bacilli, a carrier.

Disclosed herein are consortiums (a mixture of two or more distinct strains of bacteria) of bacteria. In particular, the present disclosure is directed to compositions containing one or more bacterial microorganisms from the bacterial consortia as disclosed herein, particularly in Table 1, Table 2, Table 3 or mixtures thereof. In some aspects, the composition will include two or more bacterial strains from those listed in Table 1, Table 2, Table 3 or mixtures thereof. The bacterial microorganisms can be characterized by an identifying 16S ribosomal gene sequence corresponding to, and at and least 97% identical to one or more of SEQ ID NOs: 1-8. In some aspects, the bacterial microorganisms can be characterized by an identifying 16S ribosomal gene sequence corresponding to, and at and least 97% identical to one or more of SEQ ID NOs: 1-31. In some aspects, the consortium of bacteria comprises two or more strains of bacterium having a 16S rDNA sequence comprising one or more of SEQ ID NOs: 1-31. In some aspects, the consortium of bacteria comprises three or more strains of bacterium having a 16S rDNA sequence comprising one or more of SEQ ID NOs: 1-31. In some aspects, the consortium of bacteria comprises four or more strains of bacterium having a 16S rDNA sequence comprising one or more of SEQ ID NOs: 1-31. In some aspects, the consortium of bacteria comprises five or more strains of bacterium having a 16S rDNA sequence comprising one or more of SEQ ID NOs: 1-31. In some aspects, the consortium of bacteria comprises six or more strains of bacterium having a 16S rDNA sequence comprising one or more of SEQ ID NOs: 1-31, respectively. In some aspects, the consortium of bacteria comprises seven or more strains of bacterium having a 16S rDNA sequence comprising one or more of SEQ ID NOs: 1-31. In some aspects, the consortium of bacteria comprises eight or more strains of bacterium having a 16S rDNA sequence comprising one or more of SEQ ID NOs: 1-31, respectively.

In some aspects, the consortium of bacteria comprises two or more strains of bacterium having a 16S rDNA sequence comprising SEQ ID NOs: 1, 2, 3, 4, 5, 6, 7, or 8 in the absence of any other strain of bacterium. In some aspects, the consortium of bacteria comprises two or more strains of bacterium having a 16S rDNA sequence comprising one or more of SEQ ID NOs: 1-31 in the absence of any other strain of bacterium. In some aspects, the consortium of bacteria comprises three or more strains of bacterium having a 16S rDNA sequence comprising one or more of SEQ ID NOs: 1, 2, 3, 4, 5, 6, 7, or 8 or SEQ ID NOs: 1-31 in the absence of any other strain of bacterium. In some aspects, the consortium of bacteria comprises four strains of bacterium having a 16S rDNA sequence comprising one or more of SEQ ID NOs: 1, 2, 3, 4, 5, 6, 7, or 8 or SEQ ID NOs: 1-31 in the absence of any other strain of bacterium. In some aspects, the consortium of bacteria comprises five strains of bacterium having a 16S rDNA sequence comprising one or more of SEQ ID NOs: 1, 2, 3, 4, 5, 6, 7, or 8 or SEQ ID NOs: 1-31 in the absence of any other strain of bacterium. In some aspects, the consortium of bacteria comprises five strains of bacterium having a 16S rDNA sequence comprising one or more of SEQ ID NOs: 1, 2, 3, 4, 5, 6, 7, or 8 or SEQ ID NOs: 1-31 in the absence of any other strain of bacterium. In some aspects, the consortium of bacteria comprises six strains of bacterium having a 16S rDNA sequence comprising one or more of SEQ ID NOs: 1, 2, 3, 4, 5, 6, 7, or 8 or SEQ ID NOs: 1-31 in the absence of any other strain of bacterium. In some aspects, the consortium of bacteria comprises seven strains of bacterium having a 16S rDNA sequence comprising one or more of SEQ ID NOs: 1, 2, 3, 4, 5, 6, 7, or 8 or SEQ ID NOs: 1-31 in the absence of any other strain of bacterium. In some aspects, the consortium of bacteria comprises eight strains of bacterium having a 16S rDNA sequence comprising one or more of SEQ ID NOs: 1, 2, 3, 4, 5, 6, 7, or 8 or SEQ ID NOs: 1-31 in the absence of any other strain of bacterium.

In some aspects, the consortium of bacteria comprises up to eight of the bacterial strains listed in Table 1, Table 2 or Table 3. In some aspects, the consortium of bacteria comprises two or more of the bacterial strains of Table 1. In some aspects, the consortium of bacteria comprises two or more of the bacterial strains of Table 2. In some aspects, the consortium of bacteria comprises two or more of the bacterial strains of Table 3. In some aspects, the consortium of bacteria comprises one or more bacterial strains listed in Table 1, and one or more bacterial strains listed in Table 2. In some aspects, the consortium of bacteria comprises one or more bacterial strains listed in Table 1, one or more bacterial strains listed in Table 2, and one or more bacterial strains listed in Table 3. In some aspects, the various bacteria in the consortia can be identified by their 16S ribosomal gene sequences. In some aspects, the consortium of bacteria comprises up to eight of the bacterial strains listed in Table 1, one or more bacterial strains listed in Table 2, and one or more bacterial strains listed in Table 3.

In some aspects, the compositions or the consortium of bacteria disclosed herein can reduce adiposity in a subject when colonized in the subject to the same degree as a complex microbial community that comprises a of bacteria that comprises the six strains of bacterium having a 16S rDNA sequence comprising one or more of SEQ ID NOs: 1, 2, 3, 4, 5, or 6, and one or more additional bacteria. In some aspects, the one or more additional bacteria can be any of the bacteria listed in Table 1, Table 2, Table 3 or a mixture thereof. In some aspects, the compositions or the consortium of bacteria disclosed herein can reduce adiposity in a subject when colonized in the subject to the same degree as a complex microbial community that comprises a of bacteria that comprises the six strains of bacterium having a 16S rDNA sequence comprising one or more of SEQ ID NOs: 1, 2, 3, 4, 5, 6, 7 or 8 and one or more additional bacteria. In some aspects, the one or more additional bacteria can be any of the bacteria listed in Table 1, Table 2, Table 3 or a mixture thereof. In some aspects, the compositions or the consortium of bacteria disclosed herein can reduce adiposity in a subject when colonized in the subject to the same degree as a complex microbial community that comprises a bacteria that comprises the bacterium having a 16S rDNA sequence comprising one or more of SEQ ID NOs: 1-30, or 31.

Disclosed herein is a consortium of bacteria for reducing adiposity in a subject, reducing weight gain and/or fat accumulation in a subject, lowering body fat percentage and/or reducing visceral adipose tissue (VAT) mass in a subject, decreasing blood glucose levels and/or reducing insulin resistance in a subject, inhibiting lipid absorption in a subject's small intestine, downregulating CD36 in a subject's liver or small intestine, reducing serum triglycerides and suppressing expression of lipid absorption genes within intestinal epithelia in a subject. In some aspects, the consortia of bacteria includes up to six of the bacterial strains listed in Table 1. In some aspects, a combination of any two or more of the bacterial strains of Table 1 can be used in a consortia of bacteria. In some aspects, the various bacteria in the consortia can be identified by their 16S ribosomal gene sequences. In some aspects, the consortia of bacteria includes up to six of the bacterial strains listed in Table 1, Table 2, Table 3 or mixtures thereof. In some aspects, the consortia of bacteria includes two or more of the bacterial strains listed in Table 1, Table 2, Table 3 or mixtures thereof.

TABLE 1
Bacteria in monocolonized mice.

| | | | SEQ |
|---|---|---|---|
| | | | ID |
| Strain ID | Classification | 16s sequence | NO: |
| Turicibacter | d_Bacteria; | GCGGCGAACGGGTGAGTAACACGTA | 1 |
| | p_Firmicutes; | GGTGATCTGCCCATCAGACGGGGAC | |
| | c_Bacilli; | AACGATTGGAAACGATCGCTAATAC | |
| | o_Haloplasmatales_A; | CGGATAGGACGAAAGTTTAAAGAT | |
| | f_Turicibacteraceae; | GCTCCTGGCATCACTGATGGATGA | |
| | g_Turicibacter; | GCCTGCGGCGCATTAGCTAGTTGG | |
| | s_Turicibacter | TGGGGTAAAGGCCTACCAAGGCGA | |
| | sp002311155 | CGATGCGTAGCCGACCTGAGAGGG | |
| | | TGAACGGCCACACTGGGACTGAGA | |
| | | CACGGCCCAGACTCCTACGGGAGG | |
| | | CAGCAGTAGGGAATCTTCGGCAAT | |
| | | GGGCGAAAGCCTGACCGAGCAAC | |
| | | GCCGCGTGAATGAAGAAGGCCTTC | |
| | | GGGTTGTAAAATTCTGTTATAAGG | |
| | | GAAGAAAGGTGATAGGAGGAAAT | |
| | | GACTATCAATTGACGGTACCTTAT | |
| | | GAGAAAGCCACGGCTAACTACGT | |
| | | GCCAGCAGCCGCGGTAATACGTA | |
| | | GGTGGCAAGCGTTATCCGGAATT | |
| | | ATTGGGCGTAAAGAGCGCGCAGG | |
| | | TGGTTAATTAAGTCTGATGTGAAA | |
| | | GCCCACGGCTTAACCGTGGAGGGT | |
| | | CATTGGAAACTGGTTGACTTGAGTG | |
| | | CAGAAGAGGGAAGTGGAATTCCATG | |
| | | TGTAGCGGTGAAATGCGTAGAGATA | |
| | | TGGAGGAACACCAGTGGCGAAGGC | |
| | | GGCTTCCTGGTCTGCAACTGACACTG | |
| Brevibacillus | Root[100%] | TAGCGGCGGACGGGTGAGTAACAC | 2 |
| | Bacteria[100%] | GTAGGCAACCTGCCTCTCAGACTG | |
| | Firmicutes[100%] | GGATAACATAGGGAAACTTATGCT | |
| | Bacilli[100%] | AATACCGGATAGGTTTTTGGATCG | |
| | Bacillales[100%] | CATGATCCGAAAAGAAAAGATGG | |
| | Paenibacillaceae | CTTCGGCTATCACTGGGAGATGGG | |
| | 1[100%] | CCTGCGGCGCATTAGCTAGTTGGT | |
| | Brevibacillus[100%] | GGGGTAACGGCCTACCAAGGCGA | |
| | | CGATGCGTAGCCGACCTGAGAGGG | |
| | | TGACCGGCCACACTGGGACTGAGA | |
| | | CACGGCCCAGACTCCTACGGGAGG | |
| | | CAGCAGTAGGGAATTTTCCACAAT | |
| | | GGACGAAAGTCTGATGGAGCAACG | |
| | | CCGCGTGAACGATGAAGGTCTTCG | |
| | | GATTGTAAAGTTCTGTTGTTAGGG | |
| | | ACGAATAAGTACCGTTCGAATAGG | |
| | | GCGGTACCTTGACGGTACCTGACG | |
| | | AGAAAGCCACGGCTAACTACGTGC | |
| | | CAGCAGCCGCGGTAATACGTAGGT | |
| | | GGCAAGCGTTGTCCGGATTTATTG | |
| | | GGCGTAAAGCGCGCGCAGGCGGCT | |
| | | ATGTAAGTCTGGTGTTAAAGCCCG | |
| | | GNGCTCAACCCCGGTTCGCATCGG | |
| | | AAACTGTGTAGCTTGAGTGCAGAA | |
| | | GAGGAAAGCGGTATTCCACGTGTA | |
| | | GCGGTGAAATGCGTAGAGATGTGG | |
| | | AGGAACACCAGTGGCGAAGGCGG | |
| | | CTTTC | |
| Anaerovorax | d_Bacteria; | CAGGGATAGCCATTGGAAACGATGA | 3 |
| | p_Firmicutes_A; | TTAAAACCTGATAACACCATTTGGTT | |
| | c_Clostridia; | ACATGAGCAGATGGTCAAAGATTTA | |
| | o_Peptostreptococcales; | TCGGCAAAGGATGGGCCTGCGTCTG | |
| | f_Anaerovoracaceae; | ATTAGCTAGTTGGTAAGGTAACGGC | |
| | g_Emergencia; | TTACCAAGGCGACGATCAGTAGCCG | |
| | s_Emergencia | ACCTGAGAGGGTGAACGGCCACATT | |
| | sp009936045 | GGAACTGAGACACGGTCCAAACTCC | |
| | | TACGGGAGGCAGCAGTGGGGAATAT | |
| | | TGCACAATGGGCGAAAGCCTGATGC | |
| | | AGCAACGCCGCGTGAAGGAAGAAG | |
| | | GCCTTCGGGTCGTAAACTTCTGTCCT | |
| | | TGGGGAAGAAGAACTGACGGTACCC | |
| | | AAGGAGGAAGCCCCGGCTAACTAC | |
| | | GTGCCAGCAGCCGCGGTAATACGTA | |
| | | G | |
| CXIVa | Root[100%] | CAGTTAGAAATGACTGCTAATACCG | 4 |
| | Bacteria[100%] | CATAAGACCACAAAGCCGCATGGCT | |
| | Firmicutes[100%] | RWGTGGTAAAAACTCCGGTGGTGTA | |
| | Clostridia[100%] | AGATGGGCCCGCGTCTGATTAGGTA | |
| | Clostridiales[100%] | GTTGGCGGGGTAACGGCCCACCAAG | |
| | Lachnospiraceae[100%] | CCGACGATCAGTAGCCGACCTGAGA | |
| | Clostridium XIVa[73%] | GGGTGACCGGCCACATTGGGACTGA | |
| | | GACACGGCCCAGACTCCTACGGGAG | |
| | | GCAGCAGTGGGGAATATTGCACAAT | |
| | | GGGGGAAACCCTGATGCAGCGACGC | |
| | | CGCGTGAGCGATGAAGTATTTCGGT | |
| | | ATGTAAAGCTCTATCAGCAGGGAAG | |
| | | AAAATGACGGTACCTGACTAAGAAG | |
| | | CCCCGGCTAACTACGTGCCAGCAGC | |
| | | CGCGGTAATACGTAGGGGGCAAGCG | |
| | | TTA | |
| Roseburia | Bacteria; Firmicutes; | GGGTGAGTAACGCGTGAGGAACCTG | 5 |
| | Clostridia; | CCTTGGAGTGGGGAATAACAGCTGG | |
| | Oscillospirales; | AAACAGCTGCTAATACCGCATAATA | |
| | Oscillospiraceae | TATCTGGGCCGCATGGCTCTGGATAT | |
| | | CAAAGATTTATCGCTCTGAGATGGA | |
| | | CTCGCGTCTGATTAGCTGGTTGGCGG | |
| | | GGTAACGGCCCACCAAGGCGACGAT | |
| | | CAGTAGCCGGACTGAGAGGTTGGCC | |
| | | GGCCACATTGGGACTGAGACACGGC | |
| | | CCAGACTCCTACGGGAGGCAGCAGT | |
| | | GGGGAATATTGGGCAATGGGCGCAA | |
| | | GCCTGACCCAGCAACGCCGCGTGAA | |
| | | GGAAGAAGGCTTTCGGGTTGTAAAC | |
| | | TTCTTTTGTCAGGGACGAAGCAAGT | |
| | | GACGGTACCTGACGAATAAGCCACG | |
| | | GCTAACTACGTGCCAGCAGCCGCGG | |
| | | TAATACGTAGGTGGCANNNNNTATC | |
| | | CGGATTTACTGGGTGTAAAGGGCGT | |
| | | GTAGGCGGGACTGCAAGTCAGATGT | |
| | | GAAAACCACGGGCTCAACCTGTGGC | |
| | | CTGCATTTGAAACTGTAGTTCTTGAG | |
| | | TACTGGAGAGGCAGACGGAATTCCT | |
| | | AGTGTAGCGGTGAAATGCGTAGATA | |
| | | TTAGGAGGAACACCAGTGGCGAAGG | |
| | | CGGTCTG | |
| Oscillospiraceae | Bacteria; | GGGTGAGTAACGCGTGGGCAACCTG | 6 |
| | Firmicutes; | CCCCACACAGGGGGATAACAGCCGG | |
| | Clostridia; | AAACGGCTGCTAATACCGCATAAGC | |
| | Lachnospirales; | GCACAGCTTCGCATGGAGCGGTGTG | |
| | Lachnospiraceae; | AAAAGCCTTTGGGCGGTGTGGGATG | |
| | Roseburia | GGCCCGCGTCTGATTAGCTGGTTGG | |
| | | CGGGGCAGCGGCCCACCAAGGCGAC | |
| | | GATCAGTAGCCGGCCTGAGAGGGTG | |
| | | GACGGCCACATTGGGACTGAGACAC | |
| | | GGCCCAGACTCCTGCGGGAGGCAGC | |
| | | AGTGGGGAATATTGCACAATGGGGG | |
| | | AAACCCTGATGCAGCGACGCCGCGT | |
| | | GAGCGAAGAAGTATTTCGGTATGTA | |
| | | AAGCTCTATCAGCAGGGAAGAAAAT | |
| | | GACGGTACCTGAGTAAGAAGCTCCG | |
| | | GCTAAATACGTGCCAGCAGCCGCGG | |
| | | TAATACGTATGGAGCAAGCGTTATC | |
| | | CGGATTTACTGGGTGTAAAGGGAGC | |
| | | GCAGGCGGCCAGGCAAGTCTGATGT | |
| | | GAAATACCGGGGCTCAACCCCGGAA | |
| | | CTGCATTGGAAACTGTCAGGCTGGA | |
| | | GTGTCGGAGAGGCAGGCGGAATTCC | |
| | | TGGTGTAGCGGTGAAATGCGTAGAT | |
| | | ATCAGGAGGAACACCAGTGGCGAAG | |
| | | GCGGCCTGCTG | |

TABLE 2
Bacteria in cultured community mice.

| | | | SEQ |
|---|---|---|---|
| | | | ID |
| Strain ID | Classification | 16s sequence | NO: |
| Anaerovorax | Root[100%] | CAGGGATAGCCATTGGAAACGATG | 7 |
| | Bacteria[100%] | ATTAAAACCTGATAACACCATTTGG | |
| | Firmicutes[100%] | TTACATGAGCAGATGGTCAAA | |
| | Clostridia[99%] | GATTTATCGGCAAAGGATGGGCCT | |
| | Clostridiales[99%] | GCGTCTGATTAGCTAGTTGGTAAG | |
| | Clostridiales Incertae | GTAACGGCTTACCAAGGCGACG | |
| | Sedis XIII[45%] | ATCAGTAGCCGACCTGAGAGGG | |
| | Anaerovorax[39%] | TGAACGGCCACATTGGAACTGAG | |
| | | ACACGGTCCAAACTCCTACGGGAGG | |
| | | CAGCAGTGGGGAATATTGCACAA | |
| | | TGGGCGAAAGCCTGATGCAGCAA | |
| | | CGCCGCGTGAAGGAAGAAGGCCTT | |
| | | CGGGTCGTAAACTTCTGTCCTTG | |
| | | GGGAAGAAGAACTGACGGTACC | |
| | | CAAGGAGGAAGCCCCGGCTAACTAC | |
| | | GTGCCAGCAGCCGCGGTAATACG | |
| | | TAG | |
| Eisenbergiella | Root[100%] | TACAGGGGGATAACACTTAGAAAT | 8 |
| | Bacteria[100%] | AGGTGCTAATACCGCATAAGCGCA | |
| | Firmicutes[100%] | CAGGGGCGCATGCCCCGGTGTGA | |
| | Clostridia[100%] | AAAACTCCGGTGGTATATGATGG | |
| | Clostridiales[100%] | ACCCGCGTCTGATTAGCCAGTTGG | |
| | Lachnospiraceae[100%] | CAGGGTAACGGCCTACCAAAGCGA | |
| | Eisenbergiella[67%] | CRATCARTAGCCGGCCTGAGAGGG | |
| | | CGGACGGCCACATTGGGACTGAGA | |
| | | CACGGCCCAA | |
| Clostridium | Root[100%] | CAGTTAGAAATGACTGCTAATAC | 9 |
| XIVa | Bacteria[100%] | CGCATAAGACCACAAAGCCGCAT | |
| | Firmicutes[100%] | GGCTRWGTGGTAAAAACTCCGGT | |
| | Clostridia[100%] | GGTGTAAGATGGGCCCGCGTCTG | |
| | Clostridiales[100%] | ATTAGGTAGTTGGCGGGGTAACG | |
| | Lachnospiraceae[100%] | GCCCACCAAGCCGACGATCAGTA | |
| | Clostridium XIVa[73%] | GCCGACCTGAGAGGGTGACCGG | |
| | | CCACATTGGGACTGAGACACGGC | |
| | | CCAGACTCCTACGGGAGGCAGCA | |
| | | GTGGGGAATATTGCACAATGGGG | |
| | | GAAACCCTGATGCAGCGACGCCG | |
| | | CGTGAGCGATGAAGTATTTCGGTA | |
| | | TGTAAAGCTCTATCAGCAGGGAA | |
| | | GAAAATGACGGTACCTGACTAAG | |
| | | AAGCCCCGGCTAACTACGTGCCA | |
| | | GCAGCCGCGGTAATACGTAGGGG | |
| | | GCAAGCGTTA | |
| Anaerotruncus | Root[100%] | TAATACCGCATGAGACTACAGTAC | 10 |
| | Bacteria[100%] | TACATGGTACAGTGGCCAAAGGAG | |
| | Firmicutes[98%] | CAATCCGCTGAAAGATGGGCTCGC | |
| | Clostridia[98%] | GTCCGATTAGATAGTTGGCGGGGT | |
| | Clostridiales[98%] | AACGGCCCACCAAGTCGACGATCG | |
| | Ruminococcaceae[97%] | GTAGCCGGACTGAGAGGTTGAACG | |
| | Anaerotruncus [77%] | GCCACRTTGGGACTGAGACACGGC | |
| | | CCAGACTCCTACGGGAGGCAGCAG | |
| | | TGAGGGATATTGGTCAATGGGG | |
| Oscillibacter | Root[100%] | AGTTGGAAACGACTGCTAATACCGC | 11 |
| | Bacteria[99%] | ATGATACATTTGGGTCGCATGGTCTG | |
| | Firmicutes [98%] | AATGTCAAAGATTTATCGCCGAAAG | |
| | Clostridia[97%] | ATGGCCTCGCGTCTGATTAGCTAGTT | |
| | Clostridiales[97%] | GGTGGGGTAACGGCCCACCAAGGCG | |
| | Ruminococcaceae[94%] | ACGATC | |
| | Oscillibacter[88%] | | |
| Streptococcus | Root[100%] | GCTAATACCGCATAAGAGTAGATGTT | 12 |
| | Bacteria[100%] | GCATGACATTTGCTTAAAAGGTGCAA | |
| | Firmicutes[100%] | TTGCATCACTACCAGATGGACCTGC | |
| | Bacilli[100%] | GTTGTATTAGCTAGTTGGTGGGGTAA | |
| | Lactobacillus[100%] | CGGCTCACCAAGGCGACGATACATA | |
| | Streptococcaceae[100%] | GCCGACCTGAGAGGGTGATCGGCCA | |
| | Streptococcus[100%] | CACTGGGACTGAGACACG | |

TABLE 3
Bacteria isolated from the spore forming community.

| Strain | | | SEQ ID |
|---|---|---|---|
| ID | Classification | 16s sequence | NO: |
| JRL_K | d_Bacteria; | TATTGAGAGTTTGATCCTGGCTCAGG | 13 |
| K_071 | p_Firmicutes_A; | ATGAACGCTGGCGGCGTGCTTAACAC | |
| | c_Clostridia; | ATGCAAGTCGAACGGAACACCCTTGA | |
| | o_Oscillospirales; | AGGAGTTTTCGGACAACTGAGAGGAC | |
| | f_Oscillospiraceae; | TGTTTAGTGGCGGACGGGTGAGTAAC | |
| | g_Lawsonibacter; s_ | GCGTGAGTAACCTGCCTTGGAGTGGG | |
| | | GAATAACACAAAGAAATTTGTGCTAA | |
| | | TACCGCATAATGCAGCTGAGTCGCAT | |
| | | GGCTCTGGCTGCCAAAGATTTATCGCT | |
| | | CTGAGATGGACTCGCGTCTGATTAGC | |
| | | TAGTTGGCGGGGTAACGGCCCACCAA | |
| | | GGCGACGATCAGTAGCCGGACTGAGA | |
| | | GGTTGGCCGGCCACATTGGGACTGAG | |
| | | ACACGGCCCAGACTCCTACGGGAGGC | |
| | | AGCAGTGGGGAATATTGGGCAATGGG | |
| | | CGCAAGCCTGACCCAGCAACGCCGCG | |
| | | TGAAGGAAGAAGGCTTTCGGGTTGTA | |
| | | AACTTCTTTTCTCAGGGACGAAGCAA | |
| | | GTGACGGTACCTGAGGAATAAGCCAC | |
| | | GGCTAACTACGTGCCAGCAGCCGCGG | |
| | | TAATACGTAGGTGGCAAGCGTTATCC | |
| | | GGATTTACTGGGTGTAAAGGGCGTGT | |
| | | AGGCGGGAAAGCAAGTCAGATGTGA | |
| | | AAACCATGGGCTCAACCTGTGGCCTG | |
| | | CATTTGAAACTGTTTTTCTTGAGTACT | |
| | | GGAGAGGCAGACGGAATTCCTAGTGT | |
| | | AGCGGTGAAATGCGTAGATATTAGGA | |
| | | GGAACACCAGTGGCGAAGGCGGTCTG | |
| | | CTGGACAGCAACTGACGCTGAGGCGC | |
| | | GAAAGCGTGGGGAGCAAACAGGATT | |
| | | AGATACCCTGGTAGTCCACGCTGTAA | |
| | | ACGATGGATACTAGGTGTGGGGGGAC | |
| | | TGACCCCCTCCGTGCCGCAGTTAACA | |
| | | CAATAAGTATCCCACCTGGGGAGTAC | |
| | | GATCGCAAGGTTGAAACTCAAAGGAA | |
| | | TTGACGGGGGCCCGCACAAGCGGTGG | |
| | | AGTATGTGGTTTAATTCGAAGCAACG | |
| | | CGAAGAACCTTACCAGGGCTTGACAT | |
| | | CCCACGGACAGATGTAGAGATACATC | |
| | | CTTCCCTTCGGGGACTGTGGTGACAG | |
| | | GTGGTGCATGGTTGTCGTCAGCTCGTG | |
| | | TCGTGAGATGTTGGGTTAAGTCCCGC | |
| | | AACGAGCGCAACCCTTATTGTTAGTT | |
| | | GCTACGCAAGAGCACTCTAGCGAGAC | |
| | | TGCCGTTGACAAAACGGAGGAAGGTG | |
| | | GGGACGACGTCAAATCATCATGCCCC | |
| | | TTATGTCCTGGGCCACACACGTACTAC | |
| | | AATGGTGGTCAACAGAGGGAAGCAAT | |
| | | GCCGCGAGGCGGAGCAAATCCCTAAA | |
| | | AGCCATCCCAGTTCGGATCGCAGGCT | |
| | | GCAACCCGCCTGCGTGAAGTTGGAAT | |
| | | CGCTAGTAATCGCGGATCAGCATGCC | |
| | | GCGGTGAATACGTTCCCGGGCCTTGT | |
| | | ACACACCGCCCGTCACACCATGAGAG | |
| | | TCGGGAACACCCGAAGTCCGTAGCCT | |
| | | AACCGCAAGGGGGGCGCGGCCGAAG | |
| | | GTGGGTTCGATAATTGGGGTGAAGTC | |
| | | GTAACAAGGTAGCCGTTCGAGAACGA | |
| | | GCGGCTGGATCACCTCCTTT | |
| JRL_K | d_Bacteria; | TTCGAGAGTTTGATCCTGGCTCAGGAT | 14 |
| K_081 | p_Firmicutes_A; | GAACGCTGGCGGCGTGCCTAACACAT | |
| | c_Clostridia; | GCAAGTCGAACGGGGTCATGAATTGG | |
| | o_Lachnospirales; | AAGTTTACGGATGGAAGAGACATGAC | |
| | f_Lachnospiraceae; | TTAGTGGCGGACGGGTGAGTAACGCG | |
| | g__VSOB01; s_ | TGGGTAACCTGCCGTATACAGGGGGA | |
| | | TAACACTTAGAAATAGGTGCTAACAC | |
| | | CGCATAAGCGCACGGGATTGCATGAT | |
| | | CTTGTGTGAAAATCTCCGGAGGTATA | |
| | | CGATGGGCCCGCGTCTGATTAGCCAG | |
| | | TTGGCAGGGTAACGGCCTACCAAAGC | |
| | | GACGATCAGTAGCCGGCCTGAGAGGG | |
| | | TGGACGGCCACATTGGGACTGAGACA | |
| | | CGGCCCAAACTCCTACGGGAGGCAGC | |
| | | AGTGGGGGATATTGCACAATGGGGGA | |
| | | AACCCTGATGCAGCGACGCCGCGTGA | |
| | | GTGAAGAAGTATTTCGGTATGTAAAG | |
| | | CTCTATCAGCAGGGAAGATAATGACG | |
| | | GTACCTGAGTAAGAAGCCCCGGCTAA | |
| | | CTACGTGCCAGCAGCCGCGGTAATAC | |
| | | GTAGGGGGCAAGCGTTATCCGGATTT | |
| | | ACTGGGTGTAAAGGGAGCGTAGACGG | |
| | | CAGGGCAAGTCTGATGTGAAAGCCCG | |
| | | GGGCTCAACCCCGGGACTGCATTGGA | |
| | | AACTGTCAGGCTAGAGTGCAGGAGAG | |
| | | GTGAGCGGAATTCCTAGTGTAGCGGT | |
| | | GAAATGCGTAGATATTAGGAGGAACA | |
| | | CCAGTGGCGAAGGCGGCTCACTGGAC | |
| | | TGTAACTGACGTTGAGGCTCGAAAGC | |
| | | GTGGGGAGCAAACAGGATTAGATACC | |
| | | CTGGTAGTCCACGCCGTAAACGATGA | |
| | | ATACTAGGTGTCGGTGGTTAAAGAAC | |
| | | CATCGGTGCCGCAGCAAACGCAGTAA | |
| | | GTATTCCACCTGGGGAGTACGTTCGC | |
| | | AAGAATGAAACTCAAAGGAATTGACG | |
| | | GGGACCCGCACAAGCGGTGGAGCATG | |
| | | TGGTTTAATTCGAAGCAACGCGAAGA | |
| | | ACCTTACCAAGTCTTGACATCCCCCTG | |
| | | AATAAAGGGTAATGCCGATAGCCCTT | |
| | | CGGGGCAGGGGAGACAGGTGGTGCAT | |
| | | GGTTGTCGTCAGCTCGTGTCGTGAGAT | |
| | | GTTGGGTTAAGTCCCGCAACGAGCGC | |
| | | AACCCCTATCCTTAGTAGCCAGCAGG | |
| | | TAAAGCTGGGCACTCTAGGGAGACAG | |
| | | CCGGGGATAACCCGGAGGAAGGCGG | |
| | | GGATGACGTCAAATCATCATGCCCCT | |
| | | TATGACTTGGGCTACACACGTGCTAC | |
| | | AATGGCGTGAACAAAGTGAAGCGAGC | |
| | | CAGTGATGGTAAGCAAAACACAGAAA | |
| | | GCACGTCTCAGTTCGGATTGTAGTCTG | |
| | | CAACCCGACTACATGAAGCTGGAATC | |
| | | GCTAGTAATCGCGGATCAGAATGCCG | |
| | | CGGTGAATACGTTCCCGGGTCTTGTAC | |
| | | ACACCGCCCGTCACACCATGGGAGTC | |
| | | GGAAATGCCCGAAGCCGGTGACCGAA | |
| | | CCGAAAGGAAGGAGCCGTCGAAGGC | |
| | | AGGATCGATAACTGGGGTGAAGTCGT | |
| | | AACAAGGTAGCCGTATCGGAAGGTGC | |
| | | GGCTGGATCACCTCCTTT | |
| JRL_K | d_Bacteria; | TTTATGAGAGTTTGATCCTGGCTCAGG | 15 |
| K_082 | p_Firmicutes_A; | ATGAACGCTGGCGGCGTGCCTAACAC | |
| | c_Clostridia; | ATGCAAGTCGAACGGGGCGCGCAGGC | |
| | o_Lachnospirales; | TGAGGCTTTTCGGAGCGGAAGCCTGC | |
| | f_Lachnospiraceae; | GCTGCCCAGTGGCGGACGGGTGAGTA | |
| | g_14-2; s_14-2 | ACGCGTGGGCAACCTGCCCTGCACTG | |
| | sp000403315 | GGGGACAACAGCCGGAAACGGCTGCT | |
| | | AATACCGCATATGCCCGCAGCCCCGC | |
| | | ATGGGGCGGCGGGGGAAGCTCCGGCG | |
| | | GTGCAGGATGGGCCCGCGTCTGATTA | |
| | | GCTGGTTGGCGGGGCAACGGCCCACC | |
| | | AAGGCGACGATCAGTAGCCGGCCTGA | |
| | | GAGGGTGGGCGGCCACATTGGGACTG | |
| | | AGACACGGCCCAGACTCCTACGGGAG | |
| | | GCAGCAGTGGGGGATATTGCACAATG | |
| | | GGGGGAACCCTGATGCAGCGACGCCG | |
| | | CGTGGGCGATGGAGTGCTTCGGCATG | |
| | | TAAAGCCCTATCAGCAGGGAAGAAAA | |
| | | TGACGGTACCTGACTAAGAAGCCCCG | |
| | | GCTAAATACGTGCCAGCAGCCGCGGT | |
| | | AATACGTATGGGGCAAGCGTTATCCG | |
| | | GATTTACTGGGTGTAAAGGGAGCGTA | |
| | | GGCGGCCTGGCAAGCCTGATGTGAAA | |
| | | TACCGGGGCCCAACCCCGGGGCTGCA | |
| | | TTGGGAACTGCCAGGCTGGAGTGCCG | |
| | | GAGAGGCAGGCGGAATTCCTAGTGTA | |
| | | GCGGTGAAATGCGTAGATATTAGGAG | |
| | | GAACACCAGTGGCGAAGGCGGCCTGC | |
| | | TGGACGGTGACTGACGCTGAGGCTCG | |
| | | AAAGCGTGGGGAGCAAACAGGATTA | |
| | | GATACCCTGGTAGTCCACGCCGTAAA | |
| | | CGATGAATACTAGGTGTCGGCGGGCA | |
| | | AAGCCCGCCGGTGCCGCAGCAAACGC | |
| | | AGTAAGTATTCCACCTGGGGAGTACG | |
| | | TTCGCAAGAATGAAACTCAAAGGAAT | |
| | | TGACGGGGACCCGCACAAGCGGTGGA | |
| | | GCATGTGGTTTAATTCGAAGCAACGC | |
| | | GAAGAACCTTACCAGGCCTTGACATC | |
| | | CTGCTGACGGCATATGCAATGTATGTT | |
| | | CCCTCCGGGGCAGCAGAGACAGGTGG | |
| | | TGCATGGTTGTCGTCAGCTCGTGTCGT | |
| | | GAGATGTTGGGTTAAGTCCCGCAACG | |
| | | AGCGCAACCCCTGCCCCCAGTAGCCA | |
| | | GCGCGCAAGGGCGGGCACTCTGGGGG | |
| | | GACTGCCAGGGACAACCTGGAGGAAG | |
| | | GCGGGGATGACGTCAAATCATCATGC | |
| | | CCCTTATGGCCTGGGCTACACACGTG | |
| | | CTACAATGGCGCCCACAAAGGGAGGC | |
| | | GAAATGGCGACATGGAGCAAACCCCA | |
| | | GAAAAGGCGCCCCAGTTCGGATTGCA | |
| | | GCCTGCAACTCGGCTGCATGAAGCCG | |
| | | GAATCGCTAGTAATCGCGGATCAGAA | |
| | | TGCCGCGGTGAATACGTTCCCGGGTC | |
| | | TTGTACACACCGCCCGTCACACCATG | |
| | | GGAGCCGGGAATGCCCGAAGTCAGTG | |
| | | GCCCAACCGCAAGGGGGGAGCTGCCG | |
| | | AAGGCAGGCCCGGTGACTGGGGTGAA | |
| | | GTCGTAACAAGGTAGCCGTATCGGAA | |
| | | GGTGCGGCTGGATCACCTCCTTT | |
| JRL_K | d_Bacteria; | AATTAAGAGTTTGATCCTGGCTCAGG | 16 |
| K_083 | p_Firmicutes_A; | ATGAACGCTGGCGGCGTGCCTAACAC | |
| | c_Clostridia; | ATGCAAGTCGAGCGAGAAGCTGATGA | |
| | o_Peptostreptococcales; | CAGATACTTCGGTTGAAGGAGTCAGT | |
| | f_Anaerovoracaceae; | GGAAAGCGGCGGACGGGTGAGTAAC | |
| | g_Emergencia; | GCGTAGGCAACCTGCCCTTTGCACAG | |
| | s_Emergencia | GGATAGCCATTGGAAACGATGATTAA | |
| | sp009936045 | AACCTGATAACACCATTTGGTTACAT | |
| | | GAGCAGATGGTCAAAGATTTATCGGC | |
| | | AAAGGATGGGCCTGCGTCTGATTAGC | |
| | | TAGTTGGTAAGGTAACGGCTTACCAA | |
| | | GGCGACGATCAGTAGCCGACCTGAGA | |
| | | GGGTGAACGGCCACATTGGAACTGAG | |
| | | ACACGGTCCAAACTCCTACGGGAGGC | |
| | | AGCAGTGGGGAATATTGCACAATGGG | |
| | | CGAAAGCCTGATGCAGCAACGCCGCG | |
| | | TGAAGGAAGAAGGCCTTCGGGTCGTA | |
| | | AACTTCTGTCCTTGGGGAAGAAGAAC | |
| | | TGACGGTACCCAAGGAGGAAGCCCCG | |
| | | GCTAACTACGTGCCAGCAGCCGCGGT | |
| | | AATACGTAGGGGGCAAGCGTTATCCG | |
| | | GAATTATTGGGCGTAAAGAGTACGTA | |
| | | GGTGGCAACCTAAGCGCAGGGTTTAA | |
| | | GGCAATGGCTCAACCATTGTTCGCCCT | |
| | | GCGAACTGGGATGCTTGAGTGCAGGA | |
| | | GAGGAAAGCGGAATTCCTAGTGTAGC | |
| | | GGTGAAATGCGTAGATATTAGGAGGA | |
| | | ACACCAGTGGCGAAGGCGGCTTTCTG | |
| | | GACTGTAACTGACACTGAGGTACGAA | |
| | | AGCGTGGGGAGCAAACAGGATTAGAT | |
| | | ACCCTGGTAGTCCACGCCGTAAACGA | |
| | | TGAGCACTAGGTGTCGGGGTCGCAAG | |
| | | ACTTCGGTGCCGTAGTTAACGCATTA | |
| | | AGTGCTCCGCCTGGGGAGTACGCACG | |
| | | CAAGTGTGAAACTCAAAGGAATTGAC | |
| | | GGGGACCCGCACAAGCAGCGGAGCAT | |
| | | GTGGTTTAATTCGAAGCAACGCGAAG | |
| | | AACCTTACCAGGACTTGACATCCCTCT | |
| | | GACAGACCCTTAATCGGGTTTTTCTAC | |
| | | GGACAGAGGAGACAGGTGGTGCATG | |
| | | GTTGTCGTCAGCTCGTGTCGTGAGATG | |
| | | TTGGGTTAAGTCCCGCAACGAGCGCA | |
| | | ACCCTTGCCATTAGTTGCCAGCAGTA | |
| | | AGATGGGCACTCTAGTGGGACTGCCG | |
| | | GGGACAACTCGGAGGAAGGTGGGGA | |
| | | TGACGTCAAATCATCATGCCCCTTATG | |
| | | TTCTGGGCTACACACGTGCTACAATG | |
| | | GCCGGTACAGAAAGCAGCGAAGTCGT | |
| | | GAGGCGGAGCAAATCTCGAAAACCGG | |
| | | TCCCAGTTCGGACTGCAGGCTGCAAC | |
| | | TCGCCTGCACGAAGCCGGAGTTGCTA | |
| | | GTAATCGCGGATCAGAATGCCGCGGT | |
| | | GAATGCGTTCCCGGGTCTTGTACACA | |
| | | CCGCCCGTCACACCATGGAAGTTGGG | |
| | | GGTGCCCAAAGCCGGCAAGGAAATAT | |
| | | GCTGTCTAAGGCAAAACCAATGACTG | |
| | | GGGTGAAGTCGTAACAAGGTAGCCGT | |
| | | ATCGGAAGGTGCGGCTGGATCACCTC | |
| | | CTTT | |
| JRL_K | d_Bacteria; | TATTGAGAGTTTGATCCTGGCTCAGG | 17 |
| K_088 | p_Firmicutes_A; | ATGAACGCTGGCGGCGTGCTTAACAC | |
| | c_Clostridia; | ATGCAAGTCGAACGGAACACCCTTGA | |
| | o_Oscillospirales; | AGGAGTTTTCGGACAACTGAGAGGAC | |
| | f_Oscillospiraceae; | TGTTTAGTGGCGGACGGGTGAGTAAC | |
| | g_Lawsonibacter; | GCGTGAGTAACCTGCCTTGGAGTGGG | |
| | s_ | GAATAACACAGAGAAATTTGTGCTAA | |
| | | TACCGCATAATGCAGCTGAGTCGCAT | |
| | | GGCTCTGGCTGCCAAAGATTTATCGCT | |
| | | CTGAGATGGACTCGCGTCTGATTAGC | |
| | | TAGTTGGCGGGGTAACGGCCCACCAA | |
| | | GGCGACGATCAGTAGCCGGACTGAGA | |
| | | GGTTGGCCGGCCACATTGGGACTGAG | |
| | | ACACGGCCCAGACTCCTACGGGAGGC | |
| | | AGCAGTGGGGAATATTGGGCAATGGG | |
| | | CGCAAGCCTGACCCAGCAACGCCGCG | |
| | | TGAAGGAAGAAGGCTTTCGGGTTGTA | |
| | | AACTTCTTTTCTCAGGGACGAAGCAA | |
| | | GTGACGGTACCTGAGGAATAAGCCAC | |
| | | GGCTAACTACGTGCCAGCAGCCGCGG | |
| | | TAATACGTAGGTGGCAAGCGTTATCC | |
| | | GGATTTACTGGGTGTAAAGGGCGTGT | |
| | | AGGCGGGAAAGCAAGTCAGATGTGA | |
| | | AAACCATGGGCTCAACCTGTGGCCTG | |
| | | CATTTGAAACTGTTTTTCTTGAGTACT | |
| | | GGAGAGGCAGACGGAATTCCTAGTGT | |
| | | AGCGGTGAAATGCGTAGATATTAGGA | |
| | | GGAACACCAGTGGCGAAGGCGGTCTG | |
| | | CTGGACAGCAACTGACGCTGAGGCGC | |
| | | GAAAGCGTGGGGAGCAAACAGGATT | |
| | | AGATACCCTGGTAGTCCACGCTGTAA | |
| | | ACGATGGATACTAGGTGTGGGGGGAC | |
| | | TGACCCCCTCCGTGCCGCAGTTAACA | |
| | | CAATAAGTATCCCACCTGGGGAGTAC | |
| | | GATCGCAAGGTTGAAACTCAAAGGAA | |
| | | TTGACGGGGGCCCGCACAAGCGGTGG | |
| | | AGTATGTGGTTTAATTCGAAGCAACG | |
| | | CGAAGAACCTTACCAGGGCTTGACAT | |
| | | CCCACGGACAGATGTAGAGATACATC | |
| | | CTTCCCTTCGGGGACTGTGGTGACAG | |
| | | GTGGTGCATGGTTGTCGTCAGCTCGTG | |
| | | TCGTGAGATGTTGGGTTAAGTCCCGC | |
| | | AACGAGCGCAACCCTTATTGTTAGTT | |
| | | GCTACGCAAGAGCACTCTAGCGAGAC | |
| | | TGCCGTTGACAAAACGGAGGAAGGTG | |
| | | GGGACGACGTCAAATCATCATGCCCC | |
| | | TTATGTCCTGGGCCACACACGTACTAC | |
| | | AATGGTGGTCAACAGAGGGAAGCAAT | |
| | | GCCGCGAGGCGGAGCAAATCCCTAAA | |
| | | AGCCATCCCAGTTCGGATCGCAGGCT | |
| | | GCAACCCGCCTGCGTGAAGTTGGAAT | |
| | | CGCTAGTAATCGCGGATCAGCATGCC | |
| | | GCGGTGAATACGTTCCCGGGCCTTGT | |
| | | ACACACCGCCCGTCACACCATGAGAG | |
| | | TCGGGAACACCCGAAGTCCGTAGCCT | |
| | | AACCGCAAGGGGGGCGCGGCCGAAG | |
| | | GTGGGTTCGATAATTGGGGTGAAGTC | |
| | | GTAACAAGGTAGCCGTTCGAGAACGA | |
| | | GCGGCTGGATCACCTCCTTT | |
| JRL_K | d_Bacteria; | ATGAGAGTTTGATCCTGGCTCAGGAT | 18 |
| K_096 | p_Firmicutes_A; | GAACGCTGGCGGCGTGCTTAACACAT | |
| | c_Clostridia; | GCAAGTCGAACGAAGCACTGAAAGCA | |
| | o_Lachnospirales; | GATTACTTCGGTTTGAAGCTTTCTTTG | |
| | f_Lachnospiraceae; | ACTGAGTGGCGGACGGGTGAGTAACG | |
| | g_Anaerosacchariphilus; | CGTGGGTAACCTGCCTCATACAGGGG | |
| | s_Anaerosacchariphilus | GATAACAGTTAGAAATGACTGCTAAT | |
| | sp011959465 | ACCGCATAAGCGCACGGTTTCGCATG | |
| | | GAACAGTGTGAAAAACTCCGGTGGTA | |
| | | TGGGATGGACCCGCGTCTGATTAGCT | |
| | | GGTTGGCGGGGTAACGGCCCACCAAG | |
| | | GCGACGATCAGTAGCCGGCCTGAGAG | |
| | | GGTGAACGGCCACATTGGGACTGAGA | |
| | | CACGGCCCAAACTCCTACGGGAGGCA | |
| | | GCAGTGGGGAATATTGCACAATGGGG | |
| | | GAAACCCTGATGCAGCGACGCCGCGT | |
| | | GAAGGAAGAAGTATTTCGGTATGTAA | |
| | | ACTTCTATCAGCAGGGAAGAAAATGA | |
| | | CGGTACCTGACTAAGAAGCCCCGGCT | |
| | | AACTACGTGCCAGCAGCCGCGGTAAT | |
| | | ACGTAGGGGGCAAGCGTTATCCGGAT | |
| | | TTACTGGGTGTAAAGGGAGCGTAGGC | |
| | | GGCGATGCAAGTCAGAAGTGAAAGCC | |
| | | CGGGGCTCAACTCCGGGACTGCTTTT | |
| | | GAAACTGTGTTGCTAGATTGCAGGAG | |
| | | AGGTAAGTGGAATTCCTAGTGTAGCG | |
| | | GTGAAATGCGTAGATATTAGGAGGAA | |
| | | CACCAGTGGCGAAGGCGGCTTACTGG | |
| | | ACTGTAAATGACGCTGAGGCTCGAAA | |
| | | GCGTGGGGAGCAAACAGGATTAGATA | |
| | | CCCTGGTAGTCCACGCCGTAAACGAT | |
| | | GAATACTAGGTGTCAGGGAGCAAGGC | |
| | | TCTTTGGTGCCGCAGCAAACGCAGTA | |
| | | AGTATTCCACCTGGGGAGTACGTTCG | |
| | | CAAGAATGAAACTCAAAGGAATTGAC | |
| | | GGGGACCCGCACAAGCGGTGGAGCAT | |
| | | GTGGTTTAATTCGAAGCAACGCGAAG | |
| | | AACCTTACCAAGTCTTGACATCCGGA | |
| | | TGACCGGAACTTAACCGTTCCTTCCCT | |
| | | TCGGGGCATCCGAGACAGGTGGTGCA | |
| | | TGGTTGTCGTCAGCTCGTGTCGTGAGA | |
| | | TGTTGGGTTAAGTCCCGCAACGAGCG | |
| | | CAACCCTTATCTTTAGTAGCCAGCGG | |
| | | AAGAAGCCGGGCACTCTAGAGAGACT | |
| | | GCCAGGGATAACCTGGAGGAAGGTGG | |
| | | GGATGACGTCAAATCATCATGCCCCT | |
| | | TATGATTTGGGCTACACACGTGCTAC | |
| | | AATGGCGTAAACAAAGGGAAGCGAG | |
| | | CATGCGAATGTAAGCAAATCTCAAAA | |
| | | ATAACGTCTCAGTTCGGATTGTAGTCT | |
| | | GCAACTCGACTACATGAAGCTGGAAT | |
| | | CGCTAGTAATCGCGAATCAGCATGTC | |
| | | GCGGTGAATACGTTCCCGGGTCTTGT | |
| | | ACACACCGCCCGTCACACCATGGGAG | |
| | | TTGGTAACGCCCGAAGCCAGTGACCC | |
| | | AACCGGAAACGGAGGGAGCTGTCGA | |
| | | AGGTGGGACCGATAACTGGGGTGAAG | |
| | | TCGTAACAAGGTAGCCGTATCGGAAG | |
| | | GTGCGGCTGGATCACCTCCTTT | |
| JRL_K | d_Bacteria; | GCGGCGAACGGGTGAGTAACACGTAG | 19 |
| K_T8 | p_Firmicutes; | GTGATCTGCCCATCAGACGGGGACAA | |
| | c_Bacilli; | CGATTGGAAACGATCGCTAATACCGG | |
| | o_Haloplasmatales_A; | ATAGGACGAAAGTTTAAAGATGCTCC | |
| | f_Turicibacteraceae; | TGGCATCACTGATGGATGAGCCTGCG | |
| | g_Turicibacter; | GCGCATTAGCTAGTTGGTGGGGTAAA | |
| | s_Turicibacter | GGCCTACCAAGGCGACGATGCGTAGC | |
| | sp002311155 | CGACCTGAGAGGGTGAACGGCCACAC | |
| | | TGGGACTGAGACACGGCCCAGACTCC | |
| | | TACGGGAGGCAGCAGTAGGGAATCTT | |
| | | CGGCAATGGGCGAAAGCCTGACCGAG | |
| | | CAACGCCGCGTGAATGAAGAAGGCCT | |
| | | TCGGGTTGTAAAATTCTGTTATAAGG | |
| | | GAAGAAAGGTGATAGGAGGAAATGA | |
| | | CTATCAATTGACGGTACCTTATGAGA | |
| | | AAGCCACGGCTAACTACGTGCCAGCA | |
| | | GCCGCGGTAATACGTAGGTGGCAAGC | |
| | | GTTATCCGGAATTATTGGGCGTAAAG | |
| | | AGCGCGCAGGTGGTTAATTAAGTCTG | |
| | | ATGTGAAAGCCCACGGCTTAACCGTG | |
| | | GAGGGTCATTGGAAACTGGTTGACTT | |
| | | GAGTGCAGAAGAGGGAAGTGGAATTC | |
| | | CATGTGTAGCGGTGAAATGCGTAGAG | |
| | | ATATGGAGGAACACCAGTGGCGAAGG | |
| | | CGGCTTCCTGGTCTGCAACTGACACTG | |
| JRL_K | d_Bacteria; | AGCGGCGAACGGGTGAGTAACACGTA | 20 |
| K_037 | p_Firmicutes; | GGTGATCTGCCCATCAGACGGGGACA | |
| | c_Bacilli; | ACGATTGGAAACGATCGCTAATACCG | |
| | o_Haloplasmatales_A; | GATAGGACGAAAGTTTAAAGATGCTC | |
| | f_Turicibacteraceae; | CTGGCATCACTGATGGATGAGCCTGC | |
| | g_Turicibacter; | GGCGCATTAGCTAGTTGGTGGGGTAA | |
| | s_Turicibacter | AGGCCTACCAAGGCGACGATGCGTAG | |
| | sp002311155 | CCGACCTGAGAGGGTGAACGGCCACA | |
| | | CTGGGACTGAGACACGGCCCAGACTC | |
| | | CTACGGGAGGCAGCAGTAGGGAATCT | |
| | | TCGGCAATGGGCGAAAGCCTGACCGA | |
| | | GCAACGCCGCGTGAATGAAGAAGGCC | |
| | | TTCGGGTTGTAAAATTCTGTTATAAGG | |
| | | GAAGAAAGGTGATAGGAGGAAATGA | |
| | | CTATCAATTGACGGTACCTTATGAGA | |
| | | AAGCCACGGCTAACTACGTGCCAGCA | |
| | | GCCGCGGTAATACGTAGGTGGCAAGC | |
| | | GTTATCCGGAATTATTGGGCGTAAAG | |
| | | AGCGCGCAGGTGGTTAATTAAGTCTG | |
| | | ATGTGAAAGCCCACGGCTTAACCGTG | |
| | | GAGGGTCATTGGAAACTGGTTGACTT | |
| | | GAGTGCAGAAGAGGGAAGTGGAATTC | |
| | | CATGTGTAGCGGTGAAATGCGTAGAG | |
| | | ATATGGAGGAACACCAGTGGCGAAGG | |
| | | CGGCTTCCTGGTCTGCAACTGACACT | |
| JRL_K | Bacteria; | AGCGGCGGACGGGTGAGTAACGCGTG | 21 |
| K_025 | Firmicutes; | GGCAACCTGCCTTACACAGGGGGATA | |
| | Clostridia; | ACAGTTAGAAATGACTGCTAATACCG | |
| | Lachnospirales; | CATAAGACCGCAGTACCGCATGGTAC | |
| | Lachnospiraceae; | AGCGGTAAAAACTCCGGTGGTGTAAG | |
| | Lachnoclostridium | ATGGGCCCGCGTCTGATTAGGTAGTT | |
| | | GGCGGGGTAACGGCCCACCAAGCCGA | |
| | | CGATCAGTAGCCGACCTGAGAGGGTG | |
| | | ACCGGCCACATTGGGACTGAGACACG | |
| | | GCCCAGACTCCTACGGGAGGCAGCAG | |
| | | TGGGGAATATTGCACAATGGGGGAAA | |
| | | CCCTGATGCAGCGACGCCGCGTGAGC | |
| | | GATGAAGTATTTCGGTATGTAAAGCT | |
| | | CTATCAGCAGGGAAGAAAATGACGGT | |
| | | ACCTGACTAAGAAGCCCCGGCTAACT | |
| | | ACGTGCCAGCAGCCGCGGTAATACGT | |
| | | AGGGGGCAAGCGTTATCCGGATTTAC | |
| | | TGGGTGTAAAGGGAGCGTAGACGGCT | |
| | | GTGCAAGCCANATGTGAAAGCCCGGG | |
| | | GCTCAACCCCGGGACTGCATTTGGAA | |
| | | CTGTGTGGCTGGAGTGTCNGAGAGGC | |
| | | AGGCGGAATTCCTAGTGTANCGGTGA | |
| | | AATGCGTAGATATTANGAGGAACACC | |
| | | AGTGGCGAANGCGGCCTGCTGGA | |
| JRL_K | Bacteria; | TGACGAAGCTTGCTTTTGAATTCTTGT | 22 |
| K_026 | Firmicutes; | GTATCTTAGTGGCGGACGGGTGAGTA | |
| | Clostridia; | ACGCGTGAGCAACCTGCCTTTCAGAG | |
| | Clostridiales; | GGGGATAACGTTTGGAAACGAACGCT | |
| | Ruminococcaceae; “;” | AATACCGCATGAGACTACAGTACTAC | |
| | Bacteria; | ATGGTACAGTGGCCAAAGGAGCAATC | |
| | Firmicutes; | CGCTGAAAGATGGGCTCGCGTCCGAT | |
| | Clostridia; | TAGATAGTTGGCGGGGTAACAGCCCA | |
| | Oscillospirales; | CCAAGTCGACGATCGGTAGCCGGACT | |
| | Ruminococcaceae; | GAGAGGTTGAACGGCCACATTGGGAC | |
| | Incertae Sedis; | TGAGACACGGCCCAGACTCCTACGGG | |
| | | AGGCAGCAGTGAGGGATATTGGTCAA | |
| | | TGGGGGAAACCCTGAACCAGCAACGC | |
| | | CGCGTGAGGGAAGACGGTTTTCGGAT | |
| | | TGTAAACCTCTGTCCTCTGTGAAGATA | |
| | | ATGACGGTAGCAGAGGAGGAAGCTCC | |
| | | GGCTAACTACGTGCCAGCAGCCGCGG | |
| | | TAATACGTAGGGAGCAAGCGTTGTCC | |
| | | GGATTTACTGGGTGTAAAGGGTGCGT | |
| | | AGGCGGCCTTGCAAGTCAGAAGTGAA | |
| | | ATCCATGGGCTTAACCCGTGAACTGC | |
| | | TTTTGAAACTGTAGGGCTTGAGTGAA | |
| | | GTAGAGGCAGGCGGAATTCCCGGTGT | |
| | | AGCGGTGAAATGCGTAGAGATCGGGA | |
| | | GGAACACCAGTGGCGAAGGCGGCC | |
| JRL_K | Bacteria; | GAGTGGCGGACGGGTGAGTAACGCGT | 23 |
| K_029 | Firmicutes; | GGGTAACCTGCCTCATACAGGGGGAT | |
| | Clostridia; | AACAGTTAGAAATGACTGCTAATACC | |
| | Clostridiales; | GCATAAGCGCACGGTCTCGCATGGGA | |
| | Lachnospiraceae; | CAGTGTGAAAAACTCCGGTGGTATGA | |
| | “;” | GATGGACCCGCGTCTGATTAGCTGGT | |
| | Bacteria; | TGGTGAGGTAAGGGCTCACCAAGGCG | |
| | Firmicutes; | ACGATCAGTAGCCGGCCTGAGAGGGT | |
| | Clostridia; | GAACGGCCACATTGGGACTGAGACAC | |
| | Lachnospirales; | GGCCCAAACTCCTACGGGAGGCAGCA | |
| | Lachnospiraceae; | GTGGGGAATATTGCACAATGGGGGAA | |
| | GCA-900066575; | ACCCTGATGCAGCGACGCCGCGTGAA | |
| | | GGAAGAAGTATTTCGGTATGTAAACT | |
| | | TCTATCAGCAGGGAAGAAAATGACGG | |
| | | TACCTGACTAAGAAGCCCCGGCTAAC | |
| | | TACGTGCCAGCAGCCGCGGTAATACG | |
| | | TAGGGGGCAAGCGTTATCCGGATTTA | |
| | | CTGGGTGTAAAGGGAGCGTAGGCGGT | |
| | | TCAGCAAGTCAGAAGTGAAAGCCCGG | |
| | | GGCTCAACTCCGGGACTGCTTTTGAA | |
| | | ACTGTTGAACTAGATTGCAGGAGAGG | |
| | | TAAGTGGAATTCCTAGTGTAGCGGTG | |
| | | AAATGCGTAGATATTAGGAGGAACAC | |
| | | CAGTGGCGAAGGCGGCTTACTGG | |
| JRL_K | Bacteria; | TCTTAGTGGCGGACGGGTGAGTAACG | 24 |
| K_030 | Firmicutes; | CGTGGGGAACCTGCCCTGTACAGGGG | |
| | Clostridia; | GATAACACTTANAAATAGGTGCTAAT | |
| | Clostridiales; | ACCGCATAAGCGCACGGTATCGCATG | |
| | Lachnospiraceae; | ATACAATGTGAAAAACTCCGGTGGTA | |
| | “;” | CAGGATGGACCCGCGTCTGATTAGCT | |
| | Bacteria; | GGTTGGCGGGGTAACGGCCCACCAAG | |
| | Firmicutes; | GCGACCATCATTAGCCGGCCTGAGAG | |
| | Clostridia; | GGTGAACGGACACATTGGGACTGANA | |
| | Lachnospirales; | CACGGCCCAAACTCCTACGGGAGGCA | |
| | Lachnospiraceae; | GCATTGGGGAATATTGGACAATGGGG | |
| | Lachnospiraceae | GGAACCCTGATCCAGCGACGCCGCGT | |
| | NK4A136 group; | GAGTGAAGAAGTATTTCGGTATGTAA | |
| | | AGCTCTATCAGCAGGGAAGAAAATGA | |
| | | CGGAACCTGACTAAGAAGCCCCGGCT | |
| | | AACTACGTGCCAGCAGCCGCGGTAAT | |
| | | ACGTANGGGGCAAGCGTTATCCGGAT | |
| | | TTACTGGGTGTAAAGGGAGCGTAGAC | |
| | | GGTGCAACAAGTCTGATGTGAAAGGT | |
| | | CNGGGCCCAACCCCGGGACTGCATTG | |
| | | GAAACTGTTGAACTGGAATACAGGAG | |
| | | ANGTAANCGGAATTCCTANTGTANCG | |
| | | GTGAAATGCGTNNNTATTACGAGGAA | |
| | | CACCAGTGGCNAAGGCGGCTTAC | |
| JRL_K | Bacteria; | GAACGGGTGAGTAACACGTAGGTGAT | 25 |
| K_041 | Firmicutes; | CTGCCCATCAGACGGGGACAACGATT | |
| | Bacilli; | GGAAACGATCGCTAATACCGGATAGG | |
| | Erysipelotrichales; | ACGAAAGTTTAAAGATGCTCCTGGCA | |
| | Erysipelotrichaceae; | TCACTGATGGATGAGCCTGCGGCGCA | |
| | Turicibacter | TTAGCTAGTTGGTGGGGTAAAGGCCT | |
| | | ACCAAGGCGACGATGCGTAGCCGACC | |
| | | TGAGAGGGTGAACGGCCACACTGGGA | |
| | | CTGAGACACGGCCCAGACTCCTACGG | |
| | | GAGGCAGCAGTAGGGAATCTTCGGCA | |
| | | ATGGGCGAAAGCCTGACCGAGCAACG | |
| | | CCGCGTGAATGAAGAAGGCCTTCGGG | |
| | | TTGTAAAATTCTGTTATAAGGGAAGA | |
| | | AAGGTGATAGGAGGAAATGACTATCA | |
| | | ATTGACGGTACCTTATGAGAAAGCCA | |
| | | CGGCTAACTACGTGCCAGCAGCCGCG | |
| | | GTAATACGTAGGTGGCAAGCGTTATC | |
| | | CGGAATTATTGGGCGTAAAGAGCGCG | |
| | | CAGGTGGTTAATTAAGTCTGATGTGA | |
| | | AAGCCCACGGCTTAACCGTGGAGGGT | |
| | | CATTGGAAACTGGTTGACTTGAGTGC | |
| | | AGAAGAGGGAAGTGGAATTCCATGTG | |
| | | TAGCGGTGAAATGCGTAGAGATATGG | |
| | | AGGAACACCAGTGGCGAAGGCGGCTT | |
| | | CCTGGTCTGCAACTGACACTGA | |
| JRL_K | Bacteria; | TGGCGGACGGGTGAGTAACGCGTGGG | 26 |
| K_014 | Firmicutes; | CAACCTGCCCCACACAGGGGGATAAC | |
| | Clostridia; | AGCCGGAAACGGCTGCTAATACCGCA | |
| | Lachnospirales; | TAAGCGCACAGCTTCGCATGGAGCGG | |
| | Lachnospiraceae; | TGTGAAAAGCCTTTGGGCGGTGTGGG | |
| | Roseburia or | ATGGGCCCGCGTCTGATTAGCTGGTT | |
| | d_Bacteria; | GGGGGGCAGCGGCCCACCAAGGCG | |
| | p_Firmicutes_A; | ACGATCAGTAGCCGGCCTGAGAGGGT | |
| | c_Clostridia; | GGACGGCCACATTGGGACTGAGACAC | |
| | o_Lachnospirales; | GGCCCAGACTCCTGCGGGAGGCAGCA | |
| | f_Lachnospiraceae;g | GTGGGGAATATTGCACAATGGGGGAA | |
| | 14-2;s 14-2 | ACCCTGATGCAGCGACGCCGCGTGAG | |
| | | CGAAGAAGTATTTCGGTATGTAAAGC | |
| | | TCTATCAGCAGGGAAGAAAATGACGG | |
| | | TACCTGAGTAAGAAGCTCCGGCTAAA | |
| | | TACGTGCCAGCAGCCGCGGTAATACG | |
| | | TATGGAGCAAGCGTTATCCGGATTTA | |
| | | CTGGGTGTAAAGGGAGCGCAGGCGGC | |
| | | CAGGCAAGTCTGATGTGAAATACCGG | |
| | | GGCTCAACCCCGGAACTGCATTGGAA | |
| | | ACTGTCAGGCTGGAGTGTCGGAGAGG | |
| | | CAGGCGGAATTCCTGGTGTAGCGGTG | |
| | | AAATGCGTAGATATCAGGAGGAACAC | |
| | | CAGTGGCGAAGGCGGCCTGCTGGA | |
| JRL_K | Bacteria; | GGACGGGTGAGTAACGCGTAGGCAAC | 27 |
| K_016 | Firmicutes; | CTGCCCTTAGCACAGGGATAGCCATT | |
| | Clostridia; | GGAAACGATGATTAAAACCTGATAAC | |
| | Peptostreptococcales- | ACCATTTGGTTACATGAGCAGATGGT | |
| | Tissierellales; | CAAAGATTTATCGGCAAAGGATGGGC | |
| | Anaerovoracaceae; | CTGCGTCTGATTAGCTAGTTGGTAAG | |
| | [Eubacterium] | GTAACGGCTTACCAAGGCGACGATCA | |
| | nodatum group | GTAGCCGACCTGAGAGGGTGAACGGC | |
| | | CACATTGGAACTGAGACACGGTCCAA | |
| | | ACTCCTACGGGAGGCAGCAGTGGGGA | |
| | | ATATTGCACAATGGGCGAAAGCCTGA | |
| | | TGCAGCAACGCCGCGTGAAGGAAGAA | |
| | | GGCCTTCGGGTCGTAAACTTCTGTCCT | |
| | | TGGGGAAGAAGAACTGACGGTACCCA | |
| | | AGGAGGAAGCCCCGGCTAACTACGTG | |
| | | CCAGCAGCCGCGGTAATACGTAGGGG | |
| | | GCAAGCGTTATCCGGAATTATTGGGC | |
| | | GTAAAGAGTACGTAGGTGGCAACCTA | |
| | | AGCGCAGGGTTTAAGGCAATGGCTCA | |
| | | ACCATTGTTCGCCCTGCGAACTGGGA | |
| | | TGCTTGAGTGCAGGAGAGGAAAGCGG | |
| | | AATTCCTAGTGTAGCGGTGAAATGCG | |
| | | TAGATATTAGGAGGAACACCAGTGGC | |
| | | GAAGGCGGCTTTCTGGACTGT | |
| JRL_K | Bacteria; | TCGGTTGAAGGAGTCAGTGGAAAGCG | 28 |
| K_017 | Firmicutes; | GCGGACGGGTGAGTAACGCGTAGGCA | |
| | Clostridia; | ACCTGCCCTTTGCACAGGGATAGCCA | |
| | Peptostreptococcales- | TTGGAAACGATGATTAAAACCTGATA | |
| | Tissierellales; | ACACCATTTGGTTACATGAGCAGATG | |
| | Anaerovoracaceae; | GTCAAAGATTTATCGGCAAAGGATGG | |
| | [Eubacterium] | GCCTGCGTCTGATTAGCTAGTTGGTAA | |
| | nodatum group | GGTAACGGCTTACCAAGGCGACGATC | |
| | | AGTAGCCGACCTGAGAGGGTGAACGG | |
| | | CCACATTGGAACTGAGACACGGTCCA | |
| | | AACTCCTACGGGAGGCAGCAGTGGGG | |
| | | AATATTGCACAATGGGCGAAAGCCTG | |
| | | ATGCAGCAACGCCGCGTGAAGGAAGA | |
| | | AGGCCTTCGGGTCGTAAACTTCTGTCC | |
| | | TTGGGGAAGAAGAACTGACGGTACCC | |
| | | AAGGAGGAAGCCCCGGCTAACTACGT | |
| | | GCCAGCAGCCGCGGTAATACGTAGGG | |
| | | GGCAAGCGTTATCCGGAATTATTGGG | |
| | | CGTAAAGAGTACGTAGGTGGCAACCT | |
| | | AAGCGCAGGGTTTAAGGCAATGGCTC | |
| | | AACCATTGTTCGCCCTGCGAACTGGG | |
| | | ATGCTTGAGTGCAGGAGAGGAAAGCG | |
| | | GAATTCCTAGTGTAGCGGTGAAATGC | |
| | | GTAGATATTAGGAGGAACACCAGTGG | |
| | | CGAAGGCGGCTTTCTGGAC | |
| JRL_K | Bacteria; | ATTCATATTGACTGAGTGGCGGACGG | 29 |
| K_020 | Firmicutes; | GTGAGTAACGCGTGGGTAACCTGCCT | |
| | Clostridia; | CATACAGGGGGATAACAGTTAGAAAT | |
| | Lachnospirales; | GACTGCTAATACCGCATAAGCGCACA | |
| | Lachnospiraceae | GAATCGCATGATTCGGTGTGAAAAAC | |
| | | TCCGGTGGTATGAGATGGACCCGCGT | |
| | | CTGATTAGCTAGTTGGTGGGGTAAAG | |
| | | GCCTACCAAGGCGACGATCAGTAGCC | |
| | | GACCTGAGAGGGTGATCGGCCACATT | |
| | | GGGACTGAGACACGGCCCAAACTCCT | |
| | | ACGGGAGGCAGCAGTGGGGAATATTG | |
| | | CACAATGGGGGAAACCCTGATGCAGC | |
| | | AACGCCGCGTGAGTGAAGAAGTATTT | |
| | | CGATATGTAAAGCTCTATCAGCAGGG | |
| | | AAGAAGATGACGGTACCTGAGTAAGA | |
| | | AGCCCCGGCTAACTACGTGCCAGCAG | |
| | | CCGCGGTAATACGTAGGGGGCAAGCG | |
| | | TTATCCGGATTTACTGGGTGTAAAGG | |
| | | GAGCGTAGACGGCATAGCAAGTCTGA | |
| | | AGTGAAAGCCCAAGGCTCAACCATGG | |
| | | GACTGCTTTGGAAACTGTTAAGCTAG | |
| | | AGTGCTGGAGAGGTAAGCGGAATTCC | |
| | | TAGTGTAGCGGTGAAATGCGTAGATA | |
| | | TTAGGAAGAACACCAGTGGCGAANNC | |
| | | GGCTTACTGGNC | |
| JRL_K | Bacteria; | GACTGAGTGGCGGACGGGTGAGTAAC | 30 |
| K_021 | Firmicutes; | GCGTGGGCAACCTGCCCCACACAGGG | |
| | Clostridia; | GGATAACAGCCGGAAACGGCTGCTAA | |
| | Lachnospirales; | TACCGCATAAGCGCACAGCTTCGCAT | |
| | Lachnospiraceae; | GGAGCGGTGTGAAAAGCCTTTGGGCG | |
| | Roseburia or | GTGTGGGATGGGCCCGCGTCTGATTA | |
| | d_Bacteria; | GCTGGTTGGCGGGGCAGCGGCCCACC | |
| | p_Firmicutes_A; | AAGGCGACGATCAGTAGCCGGCCTGA | |
| | c_Clostridia; | GAGGGTGGACGGCCACATTGGGACTG | |
| | o_Lachnospirales; | AGACACGGCCCAGACTCCTGCGGGAG | |
| | f_Lachnospiraceae; | GCAGCAGTGGGGAATATTGCACAATG | |
| | g_14-2; s_14-2 | GGGGAAACCCTGATGCAGCGACGCCG | |
| | sp000403845 | CGTGAGCGAAGAAGTATTTCGGTATG | |
| | | TAAAGCTCTATCAGCAGGGAAGAAAA | |
| | | TGACGGTACCTGAGTAAGAAGCTCCG | |
| | | GCTAAATACGTGCCAGCAGCCGCGGT | |
| | | AATACGTATGGAGCAAGCGTTATCCG | |
| | | GATTTACTGGGTGTAAAGGGAGCGCA | |
| | | GGCGGCCAGGCAAGTCTGATGTGAAA | |
| | | TACCGGGGCTCAACCCCGGAACTGCA | |
| | | TTGGAAACTGTCAGGCTGGAGTGTCG | |
| | | GAGAGGCAGGCGGAATTCCTGGTGTA | |
| | | GCGGTGAAATGCGTAGATATCAGGAG | |
| | | GAACACCAGTGGCGAAGGCGGCCTGC | |
| | | T | |
| JRL_K | Bacteria; | AGCGGCGGACGGGTGAGTAACGCGTG | 31 |
| K_032 | Firmicutes; | GGCAACCTGCCTTACACAGGGGGATA | |
| | Clostridia; | ACAGTTAGAAATGACTGCTAATACCG | |
| | Lachnospirales; | CATAAGACCGCAGTACCGCATGGTAC | |
| | Lachnospiraceae; | AGCGGTAAAAACTCCGGTGGTGTAAG | |
| | Lachnoclostridium | ATGGGCCCGCGTCTGATTAGGTAGTT | |
| | | GGCGGGGTAACGGCCCACCAAGCCGA | |
| | | CGATCAGTAGCCGACCTGAGAGGGTG | |
| | | ACCGGCCACATTGGGACTGAGACACG | |
| | | GCCCAGACTCCTACGGGAGGCAGCAG | |
| | | TGGGGAATATTGCACAATGGGGGAAA | |
| | | CCCTGATGCAGCGACGCCGCGTGAGC | |
| | | GATGAAGTATTTCGGTATGTAAAGCT | |
| | | CTATCAGCAGGGAAGAAAATGACGGT | |
| | | ACCTGACTAAGAAGCCCCGGCTAACT | |
| | | ACGTGCCAGCAGCCGCGGTAATACGT | |
| | | AGGGGGCAAGCGTTATCCGGATTTAC | |
| | | TGGGTGTAAAGGGAGCGTANACGGCT | |
| | | GTGCAAGCCAGATGTGAAAGCCCGGG | |
| | | GCTCAACCCCGGGACTGCATTTGGAA | |
| | | CTGTGTGGCTGGAGTGTCNGAGAGGC | |
| | | AGGCGGAATTCCTAGTGTANCGGTGA | |
| | | AATGCGTANATATTAGGAGGAACACC | |
| | | AGTGGCGAAGGCGGCCTGCTGGA | |

In some aspects, any of the compositions described herein is capable of suppressing expression of lipid adsorption genes within intestinal epithelia in a subject. In some aspects, any of the compositions disclosed herein can suppress one or more lipid absorption and/or synthesis genes. In some aspects, the lipid absorption genes can be CD36, FasN, Dgat, Srepbf1, SLC27a1, and SLC27a4.

In some aspects, any of the compositions described herein are capable of inhibiting lipid absorption in a subject's small intestine.

In some aspects, any of the compositions described herein are capable of reducing weight gain in a subject.

In some aspects, any of the compositions described herein are capable of downregulating CD36 in a subject's liver or small intestine.

In some aspects, the disclosed compositions include at least two or more bacterial microorganisms identifiable by homology of at least 95, 96, 97, 98, 99 or greater percent identity to the 16S ribosomal sequences of SEQ ID NOs: 1-31. In some aspects, the 16S sequence is less than about 1.2 kb, 1.1 kb, 1.0 kb, 0.9 kb, 8 kb, 0.7 kb, 0.6 kb, 0.5 kb, 0.4 kb, 0.3 kb, 0.2 kb, or 0.1 kb and greater than about 50 nt, 0.1 kb, 2 kb, 0.3 kb, 0.4 kb, 0.5 kb, 0.6 kb, 0.7 kb, 0.8 kb, 0.9 kb, 1.0 kb, or 1.1 kb. In some aspects, the amount of 16S ribosomal sequence homology is between about 150 nt and 500 nt, for example about 250 nt. To determine the percent identity of two nucleic acids, the sequences are aligned for optimal comparison purposes (e.g., gaps can be introduced in the sequence of a first nucleic acid sequence for optimal alignment with a second nucleic acid sequence). The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences (i.e., % identity of identical positions/total # of positions times 100).

The determination of percent homology between two sequences may be accomplished using a mathematical algorithm. A preferred, non-limiting example of a mathematical algorithm utilized for the comparison of two sequences is the algorithm of Karlin and Altschul (1990) Proc. Nat'l Acad. Sci. USA 87:2264-2268, modified as in Karlin and Altschul (1993) Proc. Nat'l Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, word length=12 to obtain nucleotide sequences similar or homologous to nucleic acid molecules of the present disclosure. To obtain gapped alignments for comparison purposes, Gapped BLAST can be utilized as described in Altschul et al. (1997) Nucleic Acids Res. 25:3389-3402. When utilizing BLAST and Gapped BLAST programs, the default parameters of the respective programs (e.g., XBLAST and NBLAST) can be used. These algorithms may be used to align DNA with RNA, and in some cases may be used to align proteins with translated nucleotide sequences.

In some aspects, at least two or more bacterial microorganisms can be included in the compositions of the present disclosure. It is contemplated that where two or more bacterial microorganisms form the composition, the bacterial microorganisms may be co-cultured to produce the disclosed composition. In some aspects, the disclosed composition may be formed by combining individual cultures of the two or more strains. The bacterial microorganisms may be propagated by methods known in the art. For example, the bacterial microorganisms may be propagated in a liquid medium under anaerobic or aerobic conditions. Suitable liquid mediums used for growing microorganism include those known in the art such as Nutrient Broth, Tryptic soy agar (TSA), Schadlers, YCFA, etc. In some aspects, the composition includes the entire listing of the strains listed in Table 1. In some aspects, the compositions can further include one or more strains listed in Table 2. In some aspects, the compositions can further include one or more strains listed in Table 3. In some aspects, the composition will include one or more bacterial strains from those listed in Table 1, Table 2, Table 3 or mixtures thereof.

In some aspects, the compositions disclosed herein can comprise at least 1×10⁴ cells of each bacterial strain. In some aspects, the compositions disclosed herein can comprise at least 1×10⁵ cells of each bacterial strain. In some aspects, the compositions disclosed herein can comprise at least 1×10⁶ cells of each bacterial strain. In some aspects, the compositions disclosed herein can comprise at least 1×10⁷ cells of each bacterial strain. In some aspects, the compositions disclosed herein can comprise at least 1×10⁸ cells of each bacterial strain. In some aspects, the compositions disclosed herein can comprise at least 1×10⁹ cells of each bacterial strain. In some aspects, the compositions disclosed herein can comprise at least 1×10¹⁰ cells of each bacterial strain. In some aspects, a single dosage of any of the compositions disclosed herein can comprise between 1×10⁴ and 1×10¹⁰ cells of each bacterial strain. In some aspects, the cells of the consortia are active.

In some aspects, the compositions disclosed herein are capable of replacing microbiota of a subject with a disease or disorder associated with an imbalanced microbiota. In some aspects, the compositions disclosed herein are capable of replacing microbiota of a subject with a disease or disorder associated with a dysfunctional microbiota. In some aspects, the compositions disclosed herein are capable of replacing microbiota of a subject with a disease or disorder associated with microbiota that is decreased in functional diversity. In some aspects, the imbalanced microbiota (or dysfunctional microbiota or a microbiota that is decreased in functional diversity) can be an increase in Desulfovibrio and decrease of Clostridia. In some aspects, the imbalanced microbiota (or dysfunctional microbiota or a microbiota that is decreased in functional diversity) can be no change in (or no expansion of) Desulfovibrio and decrease of Clostridia. In some aspects, the imbalanced microbiota (or dysfunctional microbiota or a microbiota that is decreased in functional diversity) can be a decrease of Clostridia. In some aspects, the imbalanced microbiota (or dysfunctional microbiota or a microbiota that is decreased in functional diversity) can be an absence or lack of Clostridia.

In some aspects, the disease or disorder can be obesity, metabolic syndrome, insulin deficiency, insulin deficiency, insulin-resistance related disorders, glucose intolerance, diabetes, or an inflammatory bowel disease. In some aspects, the diabetes can be type II diabetes. In some aspects, the inflammatory bowel disease can be Crohn's disease or ulcerative colitis. In some aspects, insulin-resistance related disorder can be diabetes, hyperglycemia, hypertension, dyslipidemia, or cardiovascular disease. In some aspects, diabetes can be type I diabetes. In some aspects, diabetes can be type II diabetes.

In some aspects, the compositions disclosed herein can further comprising a pharmaceutically acceptable carrier. In some aspects, the compositions can also include additives. Suitable additives include substances known in the art that may support growth, production of specific metabolites by the microorganism, alter pH, enrich for target metabolites, enhance insecticidal effects, and combinations thereof. Exemplary additives include carbon sources, nitrogen sources, phosphorous sources, inorganic salt, organic acid, growth media, vitamins, minerals, acetic acid, amino acids and the like.

Examples of suitable carbon sources include, without limitation, starch, peptone, yeast extract, amino acids, sugars such as sucrose, glucose, arabinose, mannose, glucosamine, maltose, sugar cane, alfalfa extracts, molasses, rum, and the like; salts of organic acids such as acetic acid, fumaric acid, adipic acid, propionic acid, citric acid, gluconic acid, malic acid, pyruvic acid, malonic acid, isovaleric acid, valeric acid, butyric acid and the like; alcohols such as ethanol, glycerol, and the like; oil or fat such as soybean oil, rice bran oil, olive oil, corn oil, and sesame oil. The amount of the carbon source added varies according to the kind of carbon source and is typically between 1 to 100 grams per liter of medium. The weight fraction of the carbon source in the composition may be about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition. Preferably, alfalfa is contained in the medium as a major carbon source, at a concentration of about 1 to 20% (w/v). More preferably, the alfalfa is at a concentration of about 5 to 12% (w/v).

Examples of suitable nitrogen sources include, without limitation, amino acids, yeast extract, alfalfa extract, tryptone, beef extract, peptone, potassium nitrate, ammonium nitrate, ammonium chloride, ammonium sulfate, ammonium phosphate, ammonia or combinations thereof. The amount of nitrogen source varies according to the nitrogen source, typically between 0.1 to 30 grams per liter of medium. The weight fraction of the nitrogen source in the composition may be about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

Examples of suitable inorganic salts include, without limitation, potassium dihydrogen phosphate, dipotassium hydrogen phosphate, disodium hydrogen phosphate, magnesium sulfate, magnesium chloride, ferric sulfate, ferrous sulfate, ferric chloride, ferrous chloride, manganous sulfate, manganous chloride, zinc sulfate, zinc chloride, cupric sulfate, calcium chloride, sodium chloride, calcium carbonate, sodium carbonate, and combinations thereof. The weight fraction of the inorganic salt in the composition may be about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

In some aspects, the compositions of the present disclosure can further comprise acetic acid or carboxylic acid. Suitable acetic acids include any known in the art including, without limitation, formic acid, acetic acid, propionic acid, butanoic acid, isobutyric acid, 3-methyl butanoic acid, methyl acetate ethyl acetate, propyl acetate, butyl acetate, isobutyl acetate, isovaleric acid, valeric acid, butyric acid, and 2-methyl butyl acetate. In some aspects, the acetic acid is included by using vinegar. The weight fraction of the acetic acid in the composition may be about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

In some aspects, the compositions disclosed herein can be frozen. In some aspects, the bacteria can be frozen in 25% anaerobically reduced PBS+0.1% L-Cystine or 5% DMSO. The compositions of the present disclosure can be in liquid or dry form. In some aspects, the compositions disclosed herein can be a solid. In some aspects, the compositions disclosed herein can be a liquid. In some aspects, the composition can comprise an aqueous suspension of components. This aqueous suspension can be provided as a concentrated stock solution which is diluted prior to application or as a diluted solution ready-to-use. Also, the composition can be a powder, granules, dust, pellet or colloidal concentrate. Such dry forms may be formulated to dissolve immediately upon wetting or dissolve in a controlled-release, sustained-release, or other time-dependent manner. Also, the composition may be in a dry form that does not depend upon wetting or dissolving to be effective.

In some aspects, the composition of the present disclosure can comprise at least one optional excipient. Non-limiting examples of suitable excipients include antioxidants, additives, diluents, binders, fillers, buffering agents, mineral salts, pH modifying agents, disintegrants, dispersing agents, flavoring agents, nutritive agents, oncotic and osmotic agents, stabilizers, preservatives, palatability enhancers and coloring agents. The amount and types of excipients utilized to form the combination may be selected according to known principles of science.

In some aspects, the excipient can include at least one diluent. Non-limiting examples of suitable diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lacitol, mannitol, malitol, sorbitol, xylitol, maltodextrin, and trehalose.

In some aspects, the excipient can comprise a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, polypeptides, oligopeptides, and combinations thereof.

In some aspects, the excipient can include a filler. Suitable fillers include, but are not limited to, carbohydrates, inorganic compounds, and polyvinylpyrrolidone. By way of non-limiting example, the filler may be calcium sulfate, both di-and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

In some aspects, the excipient can comprise a buffering agent. Representative examples of suitable buffering agents include, but are not limited to, MOPS, HEPES, TAPS, Bicine, Tricine, TES, PIPES, MES, Tris buffers or buffered saline salts (e.g., Tris buffered saline or phosphate buffered saline). In some aspects, the excipient can include a disintegrant. Suitable disintegrants include, but are not limited to, starches such as cornstarch, potato starch, pregelatinized and modified starches thereof, sweeteners, clays, such as bentonite, microcrystalline cellulose, alginates, sodium starch glycolate, gums such as agar, guar, locust bean, karaya, pecitin, and tragacanth.

In some aspects, the excipient can include a dispersion enhancer. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose.

In some aspects, the excipient can include a lubricant. Non-limiting examples of suitable lubricants include minerals such as talc or silica; and fats such as vegetable stearin, magnesium stearate or stearic acid.

The weight fraction of the excipient(s) in the combination can be about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the combination.

In some aspects, the compositions of the present disclosure are stable at room temperature.

In some aspects, the consortia or compositions disclosed herein can be kept at a reduced temperature for storage and transportation without significantly compromising the viability of the live bacterial microorganisms. The consortia or compositions comprising the same can be refrigerated, frozen, or lyophilized. The compositions may be refrigerated at between 32° F. to 44° F.

In some aspects, the consortia or compositions comprising the same can be stored and transported in a frozen state. The live beneficial bacterial microorganisms can be reinvigorated quickly once the compositions are thawed and brought to ambient temperature, for example, with aeration and/or agitation.

In some aspects, the consortia or compositions can be stored and transported in a manner that limits their exposure to oxygen. In some aspects, live beneficial bacterial microorganisms can be reinvigorated quickly once the compositions are thawed and brought to ambient temperature, for example, with aeration and/or agitation in an anaerobic environment. In some aspects, live beneficial bacterial microorganisms can be reinvigorated quickly once the compositions are thawed and brought to ambient temperature, for example, with aeration and/or agitation in an aerobic environment.

In some aspects, the consortia can be lyophilized. The consortia can be first frozen. Water can be then removed amendments under vacuum. This process further reduces the weight of the composition for storage and transportation. The consortia of compositions comprising the same can be reconstituted and reinvigorated prior to application or administration.

In some aspects, the concentrated consortia, or compositions comprising the same can be diluted with water before application or administration. Diluted compositions can be stored for a prolonged period of time, e.g., as long as 30 days, without losing viability. To maintain the live beneficial bacterial microorganism in a substantially aerobic state, dissolved oxygen in the diluted compositions of the present disclosure are preferably kept at an optimal level. It is preferable to supply optimal amounts of oxygen to the diluted composition though slow aeration.

In some aspects, any of the composition disclosed herein can be administered in a form selected from the group consisting of powder, granules, a ready-to-use beverage, food bar, an extruded form, capsules, gel caps, and dispersible tablets.

### METHODS

Disclosed herein are methods of altering relative abundance of microbiota in a subject. In some aspects, the methods can comprise administering to the subject an effective dose of any of the compositions disclosed herein, thereby altering the relative abundance of microbiota in the subject. In some aspects, the methods can comprise administering to the subject an effective dose of a composition comprising two or more strains of bacterium having a 16S rDNA sequence comprising at least 97% sequence identity to one or more of SEQ ID NOs: 1, 2, 3, 4, 5, 6, 7, or 8 thereby altering the relative abundance of microbiota in the subject. In some aspects, the methods can comprise administering to the subject an effective dose of a composition comprising two or more strains of bacterium having a 16S rDNA sequence comprising at least 97% sequence identity to one or more of SEQ ID NOs: 1-31 thereby altering the relative abundance of microbiota in the subject. In some aspects, the relative abundance of spore-forming bacteria can be increased. In some aspects, the relative abundance of spore-forming bacteria can be replaced. In some aspects, the compositions disclosed herein can be for replacing microbiota of a subject with a disease or disorder associated with an imbalanced microbiota (or dysfunctional microbiota or a microbiota that is decreased in functional diversity).

The method of altering microbiota can also include measuring the relative abundance of one or more microbiota in a sample from a subject. As used herein, the term “relative abundance” refers to the commonality or rarity of an organism relative to other organisms in a defined location or community. For example, the relative abundance can be determined by generally measuring the presence of a particular organism compared to the total presence of organisms in a sample.

The relative abundance of microbiota can be measured directly or indirectly. Direct measurements can include culture based methods. Indirect measurements can include comparing the prevalence of a molecular indicator of identity, such as ribosomal RNA (rRNA) gene sequences, specific for an organism or group of organisms in relation to the overall sample. For example, a ratio of rRNA specific for Desulfovibrio and Clostridia in a total number of rRNA gene sequences obtained from a cecal sample can be used to determine the relative abundance of Desulfovibrio and Clostridia in the cecal sample.

As used herein, the term “microbiota” is used to refer to one or more bacterial communities that can be found or can exist (colonize) within a gastrointestinal tract of an organism. When referring to more than one microbiota, the microbiota can be of the same type (strain) or it can be a mixture of taxa. In some aspects, the methods and compositions disclosed herein that alter the relative abundance of microbiota from genera such as Clostridium in a gastrointestinal tract of a subject. The relative abundance microbiota can be altered by administering a pharmaceutical composition that includes microbiota from genera such as Clostridium or a compound that substantially increases the relative abundance of microbiota from genera such as Clostridium, or substantially decreases the relative abundance of microbiota from or orders such Desulfovibrionales. In some aspects, the methods and compositions disclosed herein that alter the relative abundance of microbiota from a class such as Clostridia in a gastrointestinal tract of a subject. In some aspects, the methods and compositions disclosed herein that alter the relative abundance of microbiota from a class such as Bacilli in a gastrointestinal tract of a subject.

In some aspects, the relative abundance of Clostridia can be increased in the subject by at least about 5%. In some aspects, the relative abundance of Clostridia can be increased in the subject by at least about 10%. In some aspects, the relative abundance of Clostridia can be increased in the subject by at least about 1%, 2%, 3%, 4%, 5%, 6%, 7%, 8%, 9%, or 10%. In some aspects, the relative abundance of at least one of species of Clostridia can be increased by 5%.

In some aspects, the methods disclosed herein can further comprise administering a second therapeutic agent to the subject. In some aspects, the second therapeutic agent can be one or more bacteriophages. In some aspects, the one or more bacteriophages can specifically target and kill Desulfovibrio. In some aspects, the second therapeutic agent can be one or more commercially available therapeutic agents that can be administered to treat obesity, type II diabetes, and/or inflammatory bowel disease. In some aspects, the second therapeutic agent can be an anti-inflammatory agent.

Disclosed herein are methods of treating a subject with obesity. In some aspects, the methods can comprise administering to the subject any of the compositions disclosed herein, wherein the relative abundance of spore-forming microbiota is increased in the subject compared to the relative abundance prior to administration. In some aspects, the methods comprise administering to the subject a composition comprising two or more strains of bacterium having a 16S rDNA sequence comprising at least 97% sequence identity to one or more of SEQ ID NOs: 1, 2, 3, 4, 5, 6, 7, or 8, wherein the relative abundance of spore-forming microbiota is increased in the subject compared to the relative abundance prior to administration. In some aspects, the methods comprise administering to the subject a composition comprising two or more strains of bacterium having a 16S rDNA sequence comprising at least 97% sequence identity to one or more of SEQ ID NOs: 1-31, wherein the relative abundance of spore-forming microbiota is increased in the subject compared to the relative abundance prior to administration.

Disclosed herein are methods of treating a subject with metabolic syndrome. In some aspects, the methods can comprise administering to the subject any of the compositions disclosed herein, wherein the relative abundance of spore-forming microbiota is increased in the subject compared to the relative abundance prior to administration. In some aspects, the methods can comprise administering to the subject a composition comprising two or more strains of bacterium having a 16S rDNA sequence comprising at least 97% sequence identity to one or more of SEQ ID NOs: 1, 2, 3, 4, 5, 6, 7, or 8, wherein the relative abundance of spore-forming microbiota is increased in the subject compared to the relative abundance prior to administration. In some aspects, the methods can comprise administering to the subject a composition comprising two or more strains of bacterium having a 16S rDNA sequence comprising at least 97% sequence identity to one or more of SEQ ID NOs: 1-31, wherein the relative abundance of spore-forming microbiota is increased in the subject compared to the relative abundance prior to administration.

Disclosed herein are methods of treating a subject with inflammatory bowel disease. In some aspects, the methods can comprise administering to the subject any of the compositions disclosed herein, wherein the relative abundance of spore-forming microbiota is increased in the subject compared to the relative abundance prior to administration. In some aspects, the methods can comprise administering to the subject a composition comprising two or more strains of bacterium having a 16S rDNA sequence comprising at least 97% sequence identity to SEQ ID NOs: 1, 2, 3, 4, 5, 6, 7, or 8, wherein the relative abundance of spore-forming microbiota is increased in the subject compared to the relative abundance prior to administration. In some aspects, the methods can comprise administering to the subject a composition comprising two or more strains of bacterium having a 16S rDNA sequence comprising at least 97% sequence identity to one or more of SEQ ID NOs: 1-31, wherein the relative abundance of spore-forming microbiota is increased in the subject compared to the relative abundance prior to administration.

Disclosed herein are methods of reducing weight gain in a subject. In some aspects, the methods can comprise administering to the subject any of the compositions disclosed herein, wherein the relative abundance of spore-forming microbiota is increased in the subject compared to the relative abundance prior to administration. In some aspects, the methods can comprise administering to the subject a composition comprising two or more strains of bacterium having a 16S rDNA sequence comprising at least 97% sequence identity to one or more of SEQ ID NOs: 1, 2, 3, 4, 5, 6, 7, or 8, wherein the relative abundance of spore-forming microbiota is increased in the subject compared to the relative abundance prior to administration. In some aspects, the methods can comprise administering to the subject a composition comprising two or more strains of bacterium having a 16S rDNA sequence comprising at least 97% sequence identity to one or more of SEQ ID NOs: 1-31, wherein the relative abundance of spore-forming microbiota is increased in the subject compared to the relative abundance prior to administration.

Disclosed herein are methods of inhibiting lipid absorption in a subject's small intestine. In some aspects, the methods can comprise administering to the subject any of the compositions disclosed herein, wherein the relative abundance of spore-forming microbiota is increased in the subject compared to the relative abundance prior to administration. In some aspects, the methods can comprise administering to the subject a composition comprising two or more strains of bacterium having a 16S rDNA sequence comprising at least 97% sequence identity to one or more of SEQ ID NOs: 1, 2, 3, 4, 5, 6, 7, or 8, wherein the relative abundance of spore-forming microbiota is increased in the subject compared to the relative abundance prior to administration. In some aspects, the methods can comprise administering to the subject a composition comprising two or more strains of bacterium having a 16S rDNA sequence comprising at least 97% sequence identity to one or more of SEQ ID NOs: 1-31, wherein the relative abundance of spore-forming microbiota is increased in the subject compared to the relative abundance prior to administration. In some aspects, any of the compositions disclosed herein can suppress one or more lipid absorption and/or synthesis genes. In some aspects, the lipid absorption genes can be CD36, FasN, Dgat, Srepbf1, SLC27a1, and SLC27a4.

Disclosed herein are methods of downregulating CD36 in a subject's liver or small intestine. In some aspects, the methods can comprise administering to the subject any of the compositions disclosed herein, wherein the relative abundance of spore-forming microbiota is increased in the subject compared to the relative abundance prior to administration. In some aspects, the methods can comprise administering to the subject a composition comprising two or more strains of bacterium having a 16S rDNA sequence comprising at least 97% sequence identity to one or more of SEQ ID NOs: 1, 2, 3, 4, 5, 6, 7, or 8, wherein the relative abundance of spore-forming microbiota is increased in the subject compared to the relative abundance prior to administration. In some aspects, the methods can comprise administering to the subject a composition comprising two or more strains of bacterium having a 16S rDNA sequence comprising at least 97% sequence identity to one or more of SEQ ID NOs: 1-31, wherein the relative abundance of spore-forming microbiota is increased in the subject compared to the relative abundance prior to administration.

Disclosed herein are methods of reducing adiposity in a subject. In some aspects, the methods can comprise administering to the subject any of the compositions disclosed herein, wherein the relative abundance of spore-forming microbiota is increased in the subject compared to the relative abundance prior to administration. In some aspects, the methods can comprise administering to the subject a composition comprising two or more strains of bacterium having a 16S rDNA sequence comprising at least 97% sequence identity to one or more of SEQ ID NOs: 1, 2, 3, 4, 5, 6, 7, or 8, wherein the relative abundance of spore-forming microbiota is increased in the subject compared to the relative abundance prior to administration. In some aspects, the methods can comprise administering to the subject a composition comprising two or more strains of bacterium having a 16S rDNA sequence comprising at least 97% sequence identity to one or more of SEQ ID NOs: 1-31, wherein the relative abundance of spore-forming microbiota is increased in the subject compared to the relative abundance prior to administration.

Disclosed herein are methods of reducing fat accumulation in a subject. In some aspects, the methods can comprise administering to the subject any of the compositions disclosed herein, wherein the relative abundance of spore-forming microbiota is increased in the subject compared to the relative abundance prior to administration. In some aspects, the methods can comprise administering to the subject a composition comprising two or more strains of bacterium having a 16S rDNA sequence comprising at least 97% sequence identity to one or more of SEQ ID NOs: 1, 2, 3, 4, 5, 6, 7, or 8, wherein the relative abundance of spore-forming microbiota is increased in the subject compared to the relative abundance prior to administration. In some aspects, the methods can comprise administering to the subject a composition comprising two or more strains of bacterium having a 16S rDNA sequence comprising at least 97% sequence identity to one or more of SEQ ID NOs: 1-31, wherein the relative abundance of spore-forming microbiota is increased in the subject compared to the relative abundance prior to administration.

Disclosed herein are methods of lowering body fat percentage and/or reducing visceral adipose tissue (VAT) mass in a subject. In some aspects, the methods can comprise administering to the subject any of the compositions disclosed herein, wherein the relative abundance of spore-forming microbiota is increased in the subject compared to the relative abundance prior to administration. In some aspects, the methods can comprise administering to the subject a composition comprising two or more strains of bacterium having a 16S rDNA sequence comprising at least 97% sequence identity to one or more of SEQ ID NOs: 1, 2, 3, 4, 5, 6, 7, or 8, wherein the relative abundance of spore-forming microbiota is increased in the subject compared to the relative abundance prior to administration. In some aspects, the methods can comprise administering to the subject a composition comprising two or more strains of bacterium having a 16S rDNA sequence comprising at least 97% sequence identity to one or more of SEQ ID NOs: 1-31, wherein the relative abundance of spore-forming microbiota is increased in the subject compared to the relative abundance prior to administration.

Disclosed herein are methods of decreasing blood glucose levels and/or reducing insulin resistance in a subject. In some aspects, the methods can comprise administering to the subject any of the compositions disclosed herein, wherein the relative abundance of spore-forming microbiota is increased in the subject compared to the relative abundance prior to administration. In some aspects, the methods can comprise administering to the subject a composition comprising two or more strains of bacterium having a 16S rDNA sequence comprising at least 97% sequence identity to one or more of SEQ ID NOs: 1, 2, 3, 4, 5, 6, 7, or 8, wherein the relative abundance of spore-forming microbiota is increased in the subject compared to the relative abundance prior to administration. In some aspects, the methods can comprise administering to the subject a composition comprising two or more strains of bacterium having a 16S rDNA sequence comprising at least 97% sequence identity to one or more of SEQ ID NOs: 1-31, wherein the relative abundance of spore-forming microbiota is increased in the subject compared to the relative abundance prior to administration.

Disclosed herein are methods of reducing serum triglycerides in a subject. In some aspects, the methods can comprise administering to the subject any of the compositions disclosed herein, wherein the relative abundance of spore-forming microbiota is increased in the subject compared to the relative abundance prior to administration. In some aspects, the methods can comprise administering to the subject a composition comprising two or more strains of bacterium having a 16S rDNA sequence comprising at least 97% sequence identity to one or more of SEQ ID NOs: 1, 2, 3, 4, 5, 6, 7, or 8, wherein the relative abundance of spore-forming microbiota is increased in the subject compared to the relative abundance prior to administration. In some aspects, the methods can comprise administering to the subject a composition comprising two or more strains of bacterium having a 16S rDNA sequence comprising at least 97% sequence identity to one or more of SEQ ID NOs: 1-31, wherein the relative abundance of spore-forming microbiota is increased in the subject compared to the relative abundance prior to administration.

In some aspects, in any of the methods disclosed herein, the subject has been identified as being in need of the treatment. In some aspects, the subject has obesity, metabolic syndrome, insulin deficiency, insulin-resistance related disorders, glucose intolerance, diabetes, or an inflammatory bowel disease. In some aspects, the inflammatory bowel disease can be Crohn's disease or ulcerative colitis. In some aspects, insulin-resistance related disorder can be diabetes, hypertension, hyperglycemia, dyslipidemia, or cardiovascular disease. In some aspects, diabetes can be type I diabetes. In some aspects, diabetes can be type II diabetes.

As used herein, the term “metabolic disorder” or “metabolic syndrome” refers to disorders, diseases, and conditions that are caused or characterized by abnormal weight gain, energy use or consumption, altered responses to ingested or endogenous nutrients, energy sources, hormones or other signaling molecules within the body or altered metabolism of carbohydrates, lipids, proteins, nucleic acids or a combination thereof. A metabolic disorder is associated with either a deficiency or excess in a metabolic pathway resulting in an imbalance in metabolism of nucleic acids, proteins, lipids, and/or carbohydrates. Factors affecting metabolism include, and are not limited to, the endocrine (hormonal) control system (e.g., the insulin pathway, the enteroendocrine hormones including GLP-1, PYY or the like), the neural control system (e.g., GLP-1 or other neurotransmitters or regulatory proteins in the brain) or the like. Some non-limiting examples can be obesity, diabetes, including type II diabetes, insulin-deficiency, insulin-resistance, insulin-resistance related disorders, glucose intolerance, syndrome X, inflammatory and immune disorders, osteoarthritis, dyslipidemia, metabolic syndrome, non-alcoholic fatty liver, abnormal lipid metabolism, cancer, neurodegenerative disorders, sleep apnea, hypertension, high cholesterol, atherogenic dyslipidemia, hyperlipidemic conditions such as atherosclerosis, hypercholesterolemia, and other coronary artery diseases in mammals, and other disorders of metabolism.

Disorders also included are conditions that occur or cluster together, and increase the risk for heart disease, stroke, diabetes, and obesity. Having just one of these conditions such as increased blood pressure, elevated insulin levels, excess body fat around the waist or abnormal cholesterol levels can increase the risk of the above mentioned diseases. In combination, the risk for coronary heart disease, stroke, insulin-resistance syndrome, and diabetes is even greater.

In some aspects, the step of administering any of the compositions disclosed herein can comprise delivering the composition to at least a stomach, a small intestine, or a large intestine of the subject. In some aspects, the composition can be administered orally.

In some aspects, the subject can be a human.

In some aspects, the cells of the consortia are active. In some aspects, the cells of the bacterial strains disclosed herein are active.

In some aspects, in any of the methods disclosed herein, the composition can comprise or consist of one or more bacterial microorganisms from the bacterial consortia as disclosed herein, particularly in Table 1, Table 2, Table 3 or mixtures thereof. In a preferred embodiment, the composition will include two or more strains of bacterium having a 16S rDNA sequence comprising one or more of SEQ ID NOs: 1, 2, 3, 4, 5, 6, 7, or 8. The bacterial microorganisms can be characterized by an identifying 16S ribosomal gene sequence corresponding to, and at and least 97% identical to SEQ ID NOs 1-8. In some aspects, the composition will include two or more strains of bacterium having a 16S rDNA sequence comprising one or more of SEQ ID NOs: 1-31. In some aspect, the bacterial microorganisms can be characterized by an identifying 16S ribosomal gene sequence corresponding to, and at and least 97% identical to one or more of SEQ ID NOs 1-31.

### KITS

In some aspects, kits are disclosed comprising one or more bacteria, strains or bacterial microorganisms capable of for reducing adiposity in a subject, reducing weight gain and/or fat accumulation in a subject, lowering body fat percentage and/or reducing visceral adipose tissue (VAT) mass in a subject, decreasing blood glucose levels and/or reducing insulin resistance in a subject, inhibiting lipid absorption in a subject's small intestine, downregulating CD36 in a subject's liver, reducing serum triglycerides and suppressing expression of lipid absorption genes within intestinal epithelia in a subject.

In some aspects, any of the disclosed bacteria, strains, bacterial microorganism or compositions can be packaged in a suitable container labeled, for example, for use as a therapy to reduce adiposity in a subject, reduce weight gain and/or fat accumulation in a subject, lower body fat percentage and/or reduce visceral adipose tissue (VAT) mass in a subject, decrease blood glucose levels and/or reduce insulin resistance in a subject, inhibit lipid absorption in a subject's small intestine, downregulate CD36 in a subject's liver, reduce serum triglycerides and suppress expression of lipid absorption genes within intestinal epithelia in a subject. Accordingly, packaged products (e.g., sterile containers containing the bacteria, strains, bacterial microorganism or compositions described herein and packaged for storage, shipment, or sale at concentrated or ready-to-use concentrations) and kits, including bacteria, strains, bacterial microorganism or compositions as described herein and instructions for use, are also within the scope of the disclosure. A product can include a container (e.g., a vial, jar, bottle, bag, or the like) containing the bacteria, strains, bacterial microorganism or compositions described herein. In addition, the kits further can include, for example, packaging materials, instructions for use, syringes, buffers or other control reagents. The product can also include a legend (e.g., a printed label or insert or other medium describing the product's use (e.g., an audio-or videotape)). The legend can be associated with the container (e.g., affixed to the container) and can describe the manner in which the bacteria, strains, bacterial microorganism or compositions therein should be prepared, stored or administered (e.g., the frequency and route of administration), indications therefor, and other uses. In some aspects, the kits can also comprise media, culturing instructions, delivery instructions, as well as instructions for preparing, storing, delivering, etc. of the bacteria, strains, bacterial microorganism or compositions.

### EXAMPLES

### Example 1: Spore-Forming Bacteria/Clostridia can Protect Against Metabolic Disease

Mice lacking spore-forming (SF) bacteria develop metabolic disease (Petersen et al. Science 2019. DOI: 10.1126/science.aat9351).

To isolate individual SF bacteria, fecal and cecal contents from one wild type (WT) Specific Pathogen Free (SPF) mouse were used, and the contents were brought into an anaerobic chamber, resuspended in 1mL of PBS+0.1% L-cystine and 3% chloroform. This mixture was incubated for 3 hours anaerobically at 37° C. After 3 hours, the mixture was removed and the chloroform was bubbled off using air and a glass pasture pipet. Two hundred μL of this mixture was gavaged into Germ Free (GF) mice in a sterile hood and then brought into an isolator bubble.

The SF community is a diverse community containing approximately 70 distinct OTUs.

Because the diverse community of SF can convey metabolic protection from a High Fat Diet (HFD), it was assessed whether individual bacteria or a reduced consortium from the SF community can also convey metabolic protection from a HFD. To determine this, pure bacterial cultures were isolated from the SF community, their genomes were sequenced, and monocolonized mice with each isolate were used to see how they impacted host metabolism and fed mice with a normal microbiome (Specific Pathogen Free, SPF) different isolates while on a HFD. The results demonstrate that different isolates from the SF community differentially modulate the host's body composition (% Fat) and metabolism (Fasting blood glucose, insulin, and triglycerides).

Creating a culture collection from the SF community. Individual SF isolates were cultured out of the intestines of the SF bacteria colonized gnotobiotic mice (Browne et al. Nature 2016). In brief, intestinal contents from SF bacteria colonized mice (made as described herein) were harvested sterilely and quickly brought into the anaerobic chamber and resuspended in 5 mL of reduced PBS+0.1% L-cystine per gram of intestinal contents. Solids were allowed to settle out and this mixture was then 10-fold serial diluted in PBS +0.1% L-cystine and plated on YCFA, Schadler, Gifu or Modified-Gifu agar plates. Individual colonies were picked and re-streaked after 48 hours. Isolates were taxonomically identified by 16s RNA Sanger sequencing.

Tables 1 and 2 show the 16S sequences from pure culture isolates derived from the SF community and their classification. Isolates have been whole genome sequenced. Table 1 shows bacteria in monocolonized mice (e.g., SEQ ID NOs: 1-6). Table 2 shows bacteria in cultured community mice (e.g., SEQ ID NOs: 7-12).

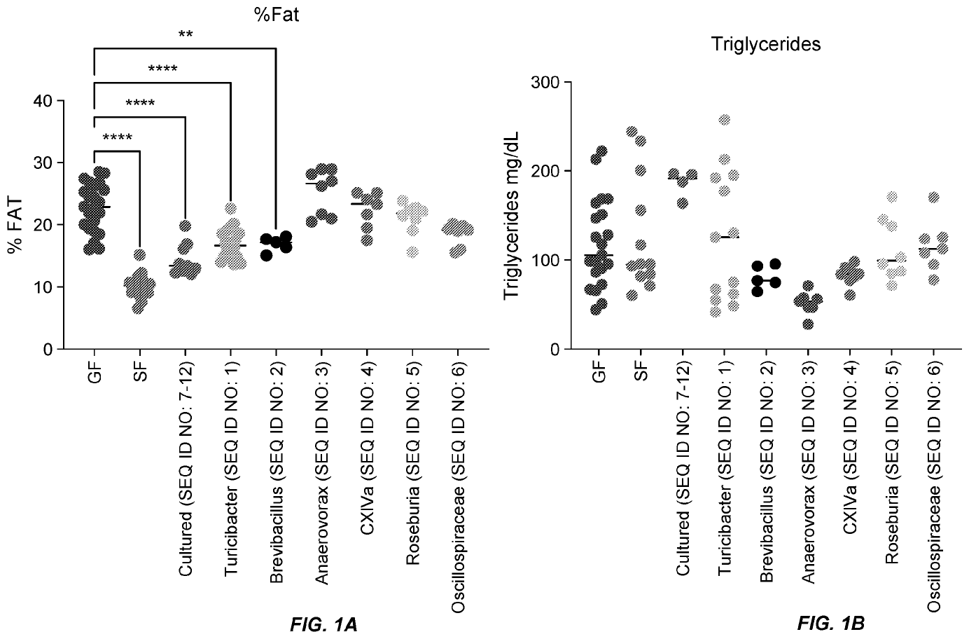

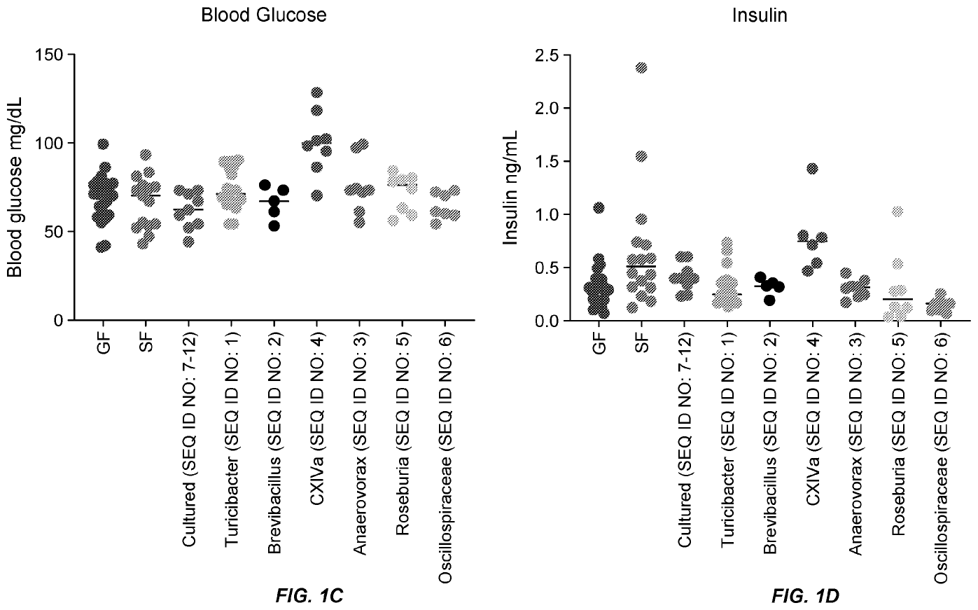

Table 3 shows the 16s sequences of isolates from the SF community that were pooled together and used to colonize mice. In FIG. 1, the group of mice with this community of bacteria are labeled “cultured”. This group of mice receiving the “cultured” community recapitulates the level of leanness seen with the whole SF community. “Cultured” and “cultured community” in this example refer to SEQ ID NOs: 13-31 (see Table 3).

Individual isolates were tested for their ability to induce leanness in a WT GF mouse by monocolonizing animals. Five week old male B6 WT GF mice were gavaged with 200 μL of pure culture in a sterile hood. Inoculated animals were put into sterile techiplast isolator cages with sterile water and normal chow. Animals lived in these cages for 8 weeks. After 8 weeks, animals were removed from the techiplast cages, weighed, NMRed, and fasted overnight. After fasting blood glucose measurements were taken and then the animals were euthanized by CO₂ and blood was drawn by cardiac puncture, allowed to clot and spun down to isolate serum for insulin and triglyceride analysis. Mice labeled turicibater_bubble have been colonized with Turicibater since birth rather than at weaning. Mice colonized with the Cultured community, Turicibacter, or Brevibacillus showed a reduced % Fat in their bodies comparable to the % Fat levels seen in animals with the whole SF community. Not all monocolonizations with bacteria result in a decrease in % Fat.

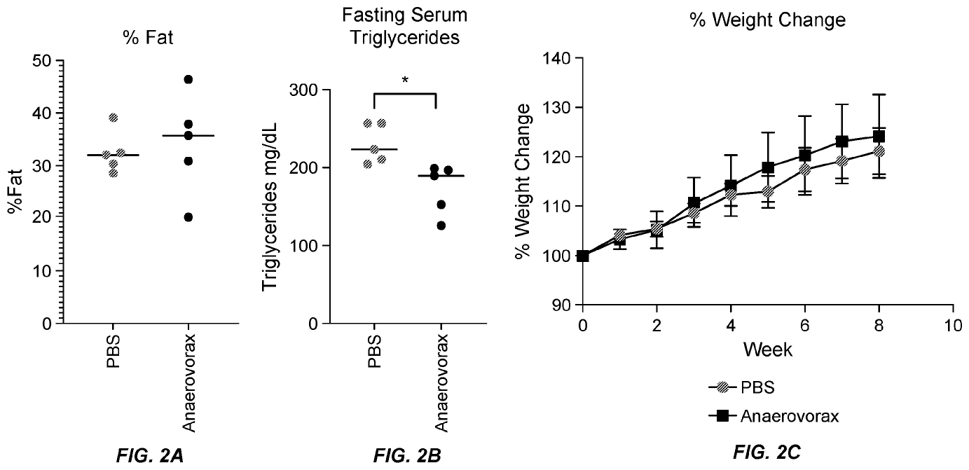

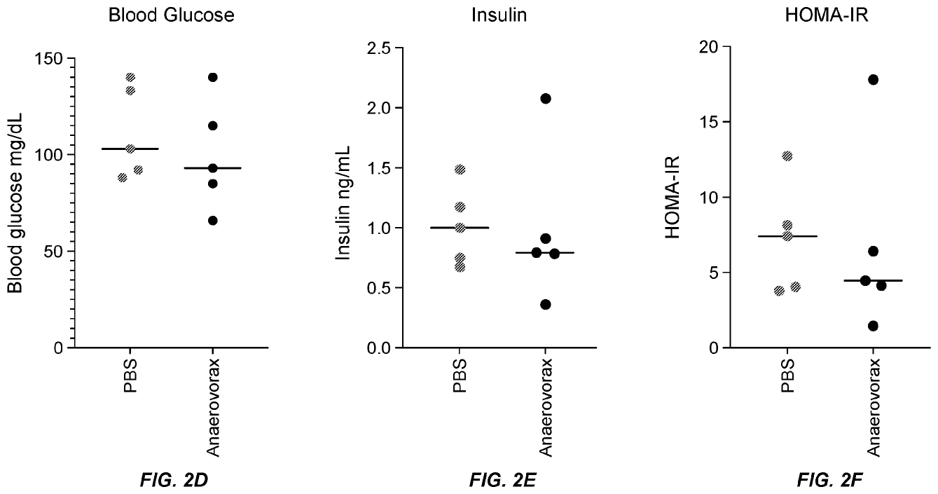

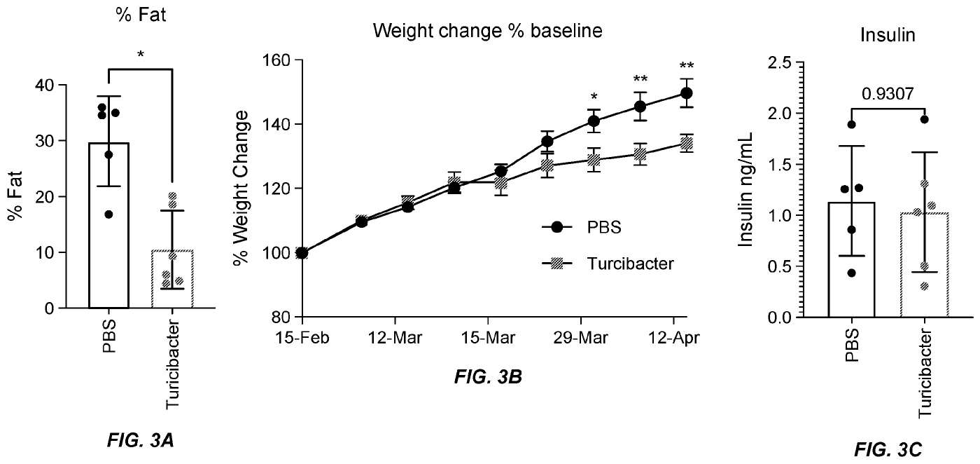

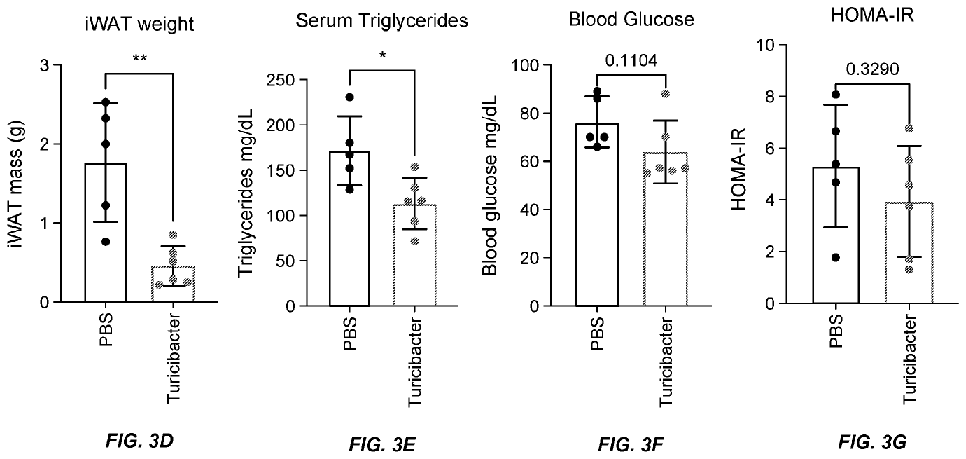

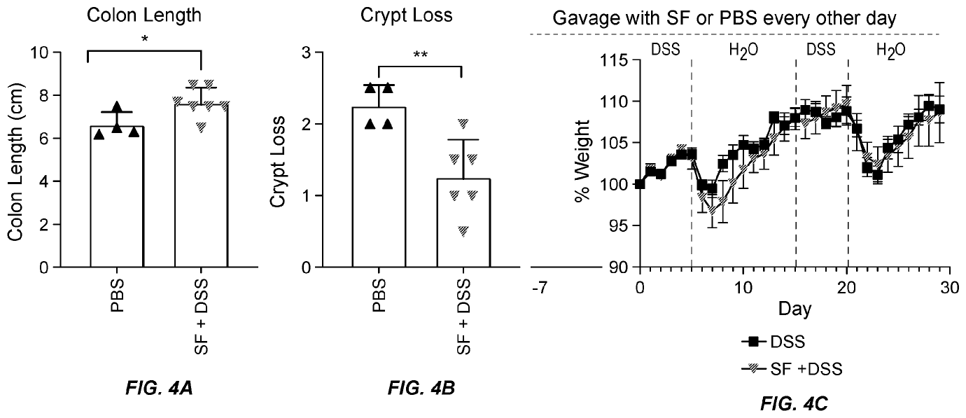

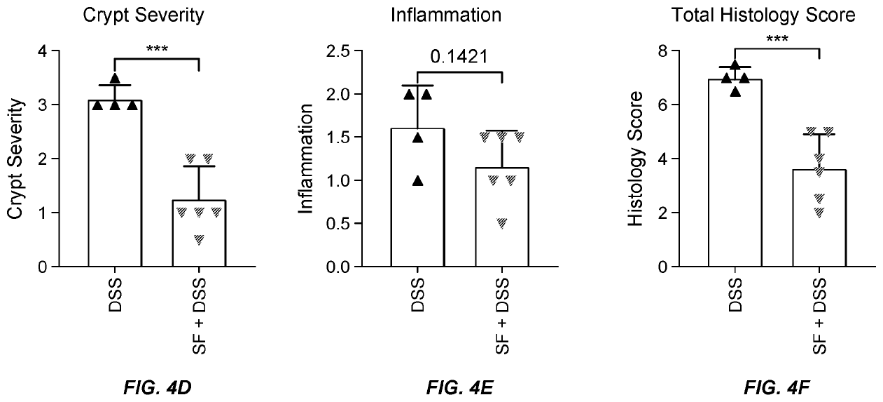

Individual isolates were tested for their ability to protect from features of metabolic disease induced by a high fat diet. Five week old male B6 WT SPF mice were put on a HFD (45% fat) at weaning and gavaged with 200 μL of culture 5 days/week or with vehicle control for 8 weeks. Anaerovorax-treated mice do not show changes in their weight or % Fat, but they did show lower fasting serum triglycerides, a marker of metabolic improvement (see, FIG. 2). Turicibacter-treated mice showed marked improvement in many metabolic parameters including weight, % Fat, and serum triglycerides (see, FIG. 3).

The mice were treated every day at a dose of 2×10\hat{ }5 CFUs/mouse of bacteria indicated in the figures including Turicubater spp. Brevibacillus, Desulfovibrio, C. anerovorax, Roseburia and Oscillspircaea. After eight weeks, animals were removed and weighed, NMRed, and fasted overnight. After fasting, blood glucose measurements were taken and then the animals were euthanized by CO₂ and blood was drawn by cardiac puncture, allowed to clot and spun down to isolate serum for insulin and triglyceride analysis.

### Example 2: Spore-Forming Bacteria/Clostridia can Protect Against Inflammatory Bowel Disease

Six week old male WT SPF mice were treated with either 200 μl/day of reduced PBS +0.1% L-cystine or intestinal contents from mice colonized with spore forming bacteria community (isolated by treating wt spf poop with chloroform as described herein; and comprising SEQ ID NOs: 1-31) resuspended in reduced PBS +0.1% L-cystine at 100 mg feces/mL. Two hundred μL/day of this SF bacteria was fed to the mice. After 7 days of this treatment with PBS or SF bacteria mice were gavaged every other day and given water containing 2.5% DSS for five days, followed by water for 10 days, followed by 2.5% DSS for five days and finished with 10 days of water. Mice were weighed daily.

1. A composition comprising bacterial strains from Clostridia or Bacilli, and a carrier.

2. The composition of claim 1, wherein the bacterial strains from Clostridia or Bacilli include an rRNA 16S having a nucleotide sequence greater than 700 nucleotides in length, wherein the nucleotide sequence is at least 97% identical to one of SEQ ID NOs: 1-31, wherein the bacterial strain from Clostridia corresponds to any one of SEQ ID NOs: 3, 4, 5, 6, 7, 8, 9, 10, 11, 13, 14, 15, 16, 17, 18, 21, 22, 23, 24, 26, 27, 28, 29, 30, or 31, or wherein the bacterial strain from Bacilli corresponds to any one of SEQ ID NOs: 1, 2, 12, 19, 20, or 25.

3.-9. (canceled)

10. The composition of claim 1, further comprising a pharmaceutically acceptable carrier.

11. The composition of claim 1, wherein the composition is frozen, a solid, or a liquid.

12.-15. (canceled)

16. The composition of claim 1, wherein the composition comprises at least 1×10⁴ cells of each bacterial strain.

17. The composition of claim 1, wherein a single dosage of the composition comprises between 1×10⁴ and 1×10¹⁰ cells of each bacterial strain.

18.-23. (canceled)

24. A method of treating a disease or disorder associated with an imbalanced microbiota in a subject, the method comprising administering to the subject an effective dose of the composition of claim 1, thereby treating a disease or disorder associated with an imbalanced microbiota in the subject.

25. (canceled)

26. (canceled)

27. The method of claim 24, further comprising administering a second therapeutic agent to the subject.

28. A method of treating a subject with obesity, the method comprising administering to the subject the composition of claim 1, wherein the relative abundance of spore forming microbiota is increased in the subject compared to the relative abundance prior to administration.

29. A method of treating a subject with metabolic syndrome, the method comprising administering to the subject the composition of claim 1, wherein the relative abundance of spore forming microbiota is increased in the subject compared to the relative abundance prior to administration.

30. A method of treating a subject with inflammatory bowel disease, the method comprising administering to the subject the composition of claim 1, wherein the relative abundance of spore forming microbiota is increased in the subject compared to the relative abundance prior to administration.

31. A method of reducing weight gain in a subject, the method comprising administering to the subject the composition of claim 1, wherein the relative abundance of spore forming microbiota is increased in the subject compared to the relative abundance prior to administration.

32. A method of inhibiting lipid absorption in a subject's small intestine, the method comprising administering to the subject the composition of claim 1, wherein the relative abundance of spore forming microbiota is increased in the subject compared to the relative abundance prior to administration.

33. A method of downregulating CD36 in a subject's liver or small intestine, the method comprising administering to the subject the composition of claim 1, wherein the relative abundance of spore forming microbiota is increased in the subject compared to the relative abundance prior to administration.

34. (canceled)

35. (canceled)

36. The method of claim 30, wherein the inflammatory bowel disease is Crohn's disease or ulcerative colitis.

37. The method of claim 24, wherein the disease or disorder is obesity, metabolic syndrome, insulin deficiency, insulin-resistance related disorder, type 2 diabetes, hyperglycemia, hypertension, dyslipidemia, glucose intolerance, an inflammatory bowel disease, or cardiovascular disease.

38. (canceled)

39. The method of claim 24, wherein the composition is administered orally,

40. The method of claim 24, wherein the relative abundance of at least one of species of Clostridia is increased by 5%.

41.-43. (canceled)

42. The method of claim 24, wherein the imbalanced microbiota is an increase in Desulfovibrio and a decrease of Clostridia.

43. The method of claim 24, wherein the imbalanced microbiota is a decrease in Clostridia and no expansion of Desulfovibrio.
